(19) United States
(12) Patent Application Publication
(10) Pub. No.: US 2025/0002929 A1
(43) Pub. Date: Jan. 2, 2025

(54) PLANT DISEASE RESISTANCE GENES AGAINST STEM RUST AND METHODS OF USE

(71) Applicant: The Sainsbury Laboratory, Norwich (GB)

(72) Inventors: Guotai Yu, Makkah Province (SA); Brande Bruce Hertel Wulff, Thuwal (SA)

(73) Assignee: The Sainsbury Laboratory, Norwich (GB)

(21) Appl. No.: 18/697,038

(22) PCT Filed: Sep. 28, 2022
(86) PCT No.: PCT/US2022/077129

§ 371 Date: Mar. 29, 2024

Related U.S. Application Data
(60) Provisional application No. 63/250,413, filed on Sep. 30, 2021.
(60) Provisional application No. 63/357,055, filed on Jun. 30, 2022.

(51) Int. Cl. C12N 15/82; A23K 10/30; A23L 7/10; C12Q 1/6874; C12Q 1/6895

(52) U.S. Cl. CPC C12N 15/8282; A23K 10/30; A23L 7/10; C12N 15/8205; C12Q 1/6874; C12Q 1/6895; C12Q 2600/13

(57) ABSTRACT

Compositions and methods and for enhancing the resistance of plants, particularly wheat plants to stem rust caused by Puccinia graminis f. sp. tritici are provided. The compositions comprise nucleic acid molecules encoding resistance (R) gene products and variants thereof and plants, seeds, and plant cells comprising such nucleic acid molecules. The methods for enhancing the resistance of a plant to stem rust comprise introducing a nucleic acid molecule encoding an R gene product into a plant cell. Additionally provided are methods for using the resistant plants in agriculture to limit stem rust.

### CROSS-REFERENCE TO RELATED APPLICATIONS

This application claims the benefit of U.S. Provisional Patent Application No. 63/250,413, filed Sep. 30, 2021 and U.S. Provisional Patent Application No. 63/357,055, filed Jun. 30, 2022; both of which are hereby incorporated herein in their entirety by reference.

### REFERENCE TO AN ELECTRONIC SEQUENCE LISTING

The contents of the electronic sequence listing (070294-0203SEQLST.xml; Size: 69,124 bytes; and Date of Creation: Sep. 20, 2022) is herein incorporated by reference in its entirety.

### FIELD OF THE INVENTION

The present invention relates to the fields of gene isolation and plant improvement, particularly to enhancing the resistance of plants to plant disease through the use of disease resistance genes.

### BACKGROUND OF THE INVENTION

Plant diseases cause significant yield losses in world-wide wheat production. Among the most damaging diseases of wheat are the rusts. Wheat stem rust caused by Puccinia graminis f. sp. tritici (Pgt) is one of the most devastating diseases affecting wheat production today. While wheat plants comprising resistance (R) genes against Pgt have proven effective in limiting the agronomic losses caused by wheat stem rust, new races of Pgt have appeared recently for which the R genes are not effective. While pesticides can be used to control wheat stem rust, pesticides are expensive and at odds with the sustainable intensification of agriculture, and in developing countries, pesticides are simply unaffordable for subsistence farmers.

The sustainable intensification of agriculture will require increased use of genetic solutions instead of chemical solutions (e.g. pesticides) to protect crops against pathogens and pests (Jones et al. (2014) Philos. T. Roy. Soc. B 369:20130087). However, traditional methods for introducing R genes typically involve long breeding timelines to break linkage to deleterious alleles of other genes. Furthermore, R genes can be overcome within a few seasons when deployed one at a time (McDonald and Linde (2002) Annu. Rev. Phytopathol. 40:349-379). Molecular cloning, however, makes it possible to avoid linkage drag and simultaneously introduce multiple R genes (Dangl et al. (2013) Science 341:746-751), which should delay resistance-breaking pathogen race evolution and thus, provide more durable resistance (McDonald and Linde (2002) Annu. Rev. Phytopathol. 40:349-379).

### BRIEF SUMMARY OF THE INVENTION

The present invention provides nucleic acid molecules for resistance (R) genes that are known to confer upon a plant resistance to at least one race of the pathogen that causes wheat stem rust, Puccinia graminis f. sp. tritici (Pgt). In one embodiment, the present invention provides nucleic acid molecules comprising the R gene, Sr43, and variants thereof including, for example, orthologs and non-naturally occurring variants. In another embodiment, the present invention provides nucleic acid molecules comprising the R gene, Sr62, and variants thereof including, for example, orthologs and non-naturally occurring variants.

The present invention further provides plants, plant cells, and seeds comprising in their genomes one or more polynucleotide constructs of the invention. The polynucleotide constructs comprise a nucleotide sequence encoding a resistance (R) protein of the present invention. Such R proteins are encoded by the R genes of the present invention. In a preferred embodiment, the plants and seeds are transgenic wheat plants and seeds that have been transformed with one or more polynucleotide constructs of the invention. Preferably, such wheat plants comprise enhanced resistance to at least one race of the pathogen, Pgt, that causes wheat stem rust when compared to the resistance of a control wheat plant that does not comprise the polynucleotide construct.

The present invention provides methods for enhancing the resistance of a plant, particularly a wheat, barley, or triticale plant, to stem rust caused by Pgt. Such methods comprise introducing into at least one plant cell a polynucleotide construct comprising a nucleotide sequence of an R gene of the present invention. In some embodiments, the polynucleotide construct or part thereof is stably incorporated into the genome of the plant cell, and in other embodiments, the polynucleotide construct is not stably incorporated into the genome of the plant cell. The methods for enhancing the resistance of a plant to stem rust can optionally further comprise regenerating the plant cell into a plant that comprises in its genome the polynucleotide construct. Preferably, such a plant comprises enhanced resistance to stem rust caused by at least one race of Pgt, relative to a control plant.

The present invention additionally provides methods for identifying a plant, particularly a wheat, barley, or triticale plant, that displays newly conferred or enhanced resistance to stem rust caused by Pgt. The methods comprise detecting in the plant the presence of at least one R gene of the present invention, particularly Sr43 and/or Sr62.

Methods of using the plants of the present invention in agricultural crop production to limit stem rust caused by Pgt are also provided. The methods comprise planting a seed produced by a plant of the present invention, wherein the seed comprises at least one R gene nucleotide sequence of the present invention. The methods further comprise growing a plant under conditions favorable for the growth and development of the plant, and optionally harvesting at least one seed or plant part from the plant.

Additionally provided are plants, plant parts, seeds, plant cells, other host cells, expression cassettes, and vectors comprising one or more of the nucleic acid molecules of the present invention.

### BRIEF DESCRIPTION OF THE DRAWINGS

FIGS. 1A-1D. Sr43 gene structure and functional confirmation by transgenics. FIG. 1A is a schematic representation of the Sr43 primary transcript which consists of 18 exons (black boxes), including 5′ and 3′ UTRs, interspersed by introns (connecting lines). The position of the start (ATG) and stop (TAG) codons are indicated by arrows. FIG. 1B is a schematic representation of the Sr43 protein, with the position of the two kinase domains and the predicted amino-acid changes caused by the EMS mutations indicated. Splice 1-4 refer to splice variants 1-4, respectively, of Sr43. FIG. 1C is a schematic representation of the 13.5 kb genomic region used for cloning into a binary construct for transformation into wheat consists of 3.2 kb of putative 5′ regulatory sequence (promoter), 7.8 kb of the coding DNA sequence (CDS)-containing genomic region from the start (ATG) to the stop (TAG) codons based on splice 1, and 2.5 kb of 3′ putative regulatory sequence (terminator). (c) The 866 amino acid sequence contains homology to a protein kinase [PKc_like (S_TKc)] and domains of unknown function (DUF) numbers 3475 and 668. FIG. 1D shows the results of disease resistance assays in which susceptible plants (Fielder) and T1 plants transformed with Sr43 (Fielder-Sr43-T1) were tested with four Pgt races from UK and Israel.

FIGS. 2A-2D. Functional validation of Sr62 by EMS mutagenesis and transformation into wheat. FIG. 2A: Structure of Sr62, with predicted nucleotide change caused by EMS-derived loss-of-function mutations. Boxes represent exons and lines represent introns. The 11.4-kb portion of the third intron excluded from the binary construct is indicated. FIG. 2B: Schematic representation of the Sr62 protein, with the position of the two kinase domains and the predicted amino-acid changes caused by the EMS mutations indicated. FIG. 2C: The Sr62 sequence used for transformation of wheat cultivar Fielder. CDS, coding DNA sequence. FIG. 2D: Reactions of three homozygous independent transgenic lines to four stem rust isolates. The copy number of the hygromycin selectable marker in T₀ plants is indicated.

FIG. 3. Functional validation by tests on Sr62 transgenics T1 plants with eight additional stem rust isolates/races.

FIG. 4. Reactions of Sr43 T₂ transgenic plants and controls to various races/isolates of the fungal agent causing stem rust. The reactions of homozygous (T₂) transgenic lines (Sr43 T₂) were compared to the reactions of null segregants (Sr43 T₂ null), Fielder, and Sr43 wild-type introgression lines (Sr43).

FIG. 5. Reactions of Sr43 T₂ transgenic plants and controls to various races/isolates of the fungal agent causing stem rust. Representative leaves from seedlings of Sr43 wild-type (Sr43) and transgenic (Sr43 T₂) lines alongside non-transgenic wild-type Fielder and null controls (Sr43 T₂ null) inoculated with Puccinia graminis f. sp. tritici isolates 14GEO189-1 (avirulent on Sr43) and 75ND717C (intermediately virulent on Sr43) are shown.

FIG. 6. Confirmation of Sr43 temperature sensitivity. The effect of temperature on Sr43-mediated resistance to Pgt isolate 69MN399 on homozygous (T₂) transgenic lines (Sr43 T₂) as compared to null segregants (Sr43 T₂ null), Fielder, and Sr43 wild-type introgression lines (Sr43).

### SEQUENCE LISTING

The nucleotide and amino acid sequences listed in the accompanying sequence listing, drawings, and those set forth hereinbelow are shown using standard letter abbreviations for nucleotide bases, and either the one-letter or three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. For the purposes of illustration, RNA sequences (e.g. transcripts) may be represented in the sequence listing as DNA sequences with “U” nucleotides in the RNA sequence represented by “T” nucleotides. The amino acid sequences follow the standard convention of beginning at the amino terminus of the sequence and proceeding forward (i.e., from left to right in each line) to the carboxy terminus.
SEQ ID NO: 1 sets forth the nucleotide sequence comprising the R gene, Sr43, from Thinopyrum ponticum.
SEQ ID NO: 2 sets forth the full-length amino acid sequence of the protein encoded by splice variant 1 (SV1) of the R gene, Sr43.
SEQ ID NO: 3 sets forth the full-length amino acid sequence of the protein encoded by splice variant 2 (SV2) of the R gene, Sr43.
SEQ ID NO: 4 sets forth the full-length amino acid sequence of the protein encoded by splice variant 3 (SV3) of the R gene, Sr43.
SEQ ID NO: 5 sets forth the full-length amino acid sequence of the protein encoded by splice variant 4 (SV4) of the R gene, Sr43.
SEQ ID NO: 6 sets forth the nucleotide sequence of the full-length coding region of the cDNA of SV1 of Sr43. If desired, a stop codon (e.g. TAA, TAG, or TGA) can be operably linked to the 3′ end of a nucleic acid molecule comprising or consisting of SEQ ID NO: 6.
SEQ ID NO: 7 sets forth the nucleotide sequence of the full-length coding region of the cDNA of SV2 of Sr43. If desired, a stop codon (e.g. TAA, TAG, or TGA) can be operably linked to the 3′ end of a nucleic acid molecule comprising or consisting of SEQ ID NO: 7.
SEQ ID NO: 8 sets forth the nucleotide sequence of the full-length coding region of the cDNA of SV3 of Sr43. If desired, a stop codon (e.g. TAA, TAG, or TGA) can be operably linked to the 3′ end of a nucleic acid molecule comprising or consisting of SEQ ID NO: 8.
SEQ ID NO: 9 sets forth the nucleotide sequence of the full-length coding region of the cDNA of SV4 of Sr43. If desired, a stop codon (e.g. TAA, TAG, or TGA) can be operably linked to the 3′ end of a nucleic acid molecule comprising or consisting of SEQ ID NO: 9.
SEQ ID NO: 10 sets forth the nucleotide sequence of the SV1 transcript of Sr43.
SEQ ID NO: 11 sets forth the nucleotide sequence of the SV2 transcript of Sr43.
SEQ ID NO: 12 sets forth the nucleotide sequence of the SV3 transcript of Sr43.
SEQ ID NO: 13 sets forth the nucleotide sequence of the SV4 transcript of Sr43.
SEQ ID NO: 14 sets forth the nucleotide sequence comprising the R gene, Sr62, from Aegilops sharonensis.
SEQ ID NO: 15 sets forth the full-length amino acid sequence of the R protein encoded by the R gene, Sr62.
SEQ ID NO: 16 sets forth the nucleotide sequence of the full-length coding region of the cDNA of Sr62. If desired, a stop codon (e.g. TAA, TAG, or TGA) can be operably linked to the 3′ end of a nucleic acid molecule comprising or consisting of SEQ ID NO: 16.
SEQ ID NO: 17 sets forth the nucleotide sequence of the transcript of Sr62.

### DETAILED DESCRIPTION OF THE INVENTION

The present inventions now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

The present invention relates to the isolation of plant resistance (R) genes, particularly R genes that confers upon a plant, particularly a wheat, barley, or triticale plant, resistance to stem rust caused by Puccinia graminis f. sp. tritici (Pgt).

The present invention provides nucleic acid molecules comprising the nucleotide sequences of R genes, particularly the nucleotide sequences of Sr43 and Sr62 and naturally occurring (e.g. orthologs and allelic variants) and synthetic or artificial (i.e. non-naturally occurring) variants thereof. Both Sr43 and Sr62 are known to confer resistance to stem rust cased by multiple races of Pgt. As disclosed hereinbelow, the present inventors identified the Sr43 gene in a wheat-Thinopyrum ponticum introgression line containing Sr43 through the use of a complexity reduction approach based on flow sorting and sequencing of mutant chromosomes, to identify induced mutations by comparison to parental chromosomes As disclosed hereinbelow, the present inventors identified the Sr62 gene through a combination of (i) mapping of Pgt resistance in a cross between Aegilops sharonensis accessions 1644 (resistant) and 2189 (susceptible) to delimit Sr62 to a genetic interval of 0.01 cM, (ii) assembly of the genome of Ae. sharonensis accession 1644 followed by projection of the genetic markers flanking Sr62 onto the genome sequence thus delimiting the Sr62 map interval to 480 kb, (iii) RNA-Seq of Ae. sharonensis accession 1644 and annotation of candidate genes in the 480 kb Sr62 interval, (iv) EMS mutagenesis of a wheat-Ae. sharonensis accession 1644 introgression line, identification of susceptible mutants, RNA-Seq of the susceptible mutants, and comparison of the RNA-Seq reads to the parental sequences to identify induced mutations.

The nucleotide sequences of the R genes of the present invention, which are also referred to herein as R gene nucleotide sequences, encode R proteins. R gene nucleotide sequences of the invention include, but are not limited to, wild-type R genes comprising a native promoter and the 3′ adjacent region comprising the coding region, cDNA sequences, and nucleotide sequences comprising only the coding region. Examples of such R gene nucleotide sequences include the nucleotide sequences of Sr43 set forth in SEQ ID NOS: 1, 6, 7, 8, 9, 10, 11, 12, and 13 and variants thereof and the nucleotide sequences of Sr62 set forth in SEQ ID NOS: 14, 16, and 17. In embodiments in which the native R gene promoter is not used to drive the expression of the nucleotide sequence encoding the R protein, a heterologous promoter can be operably linked to a nucleotide sequence encoding an R protein of the invention to drive the expression of nucleotide sequence encoding an R protein in a plant. In certain embodiments, the R genes of the present invention are capable of conferring to a plant broad-spectrum resistance to multiple races of Pgt such as, for example, the R genes Sr43 and Sr62.

Preferably, the R proteins of the invention are functional R proteins that are capable of conferring to a plant comprising the R protein enhanced resistance to stem rust caused by at least one race of Pgt. In certain embodiments, the R proteins of the present invention comprise broad-spectrum resistance to multiple races of Pgt such as, for example, the R protein encoded by Sr43 and the R protein encoded by Sr62.

The present invention further provides transgenic plants comprising a polynucleotide construct which comprise an R gene nucleotide sequence of the invention. In some embodiments, the polynucleotide construct is stably incorporated into the genome of the plant, and in other embodiments, the plant is transformed by a transient transformation method and the polynucleotide construct is not stably incorporated into the genome of the plant. Methods for both the stable and transient transformation of plants are disclosed elsewhere herein or otherwise known in the art. In a preferred embodiment of the invention, the transgenic plants are wheat plants that comprise enhanced resistance to stem rust caused by at least one race of Pgt.

In certain embodiments, a transgenic plant of the invention comprises a polynucleotide construct comprising a nucleotide sequence encoding an R protein and a heterologous promoter that is operably linked for expression of the nucleotide sequence encoding an R protein. The choice of heterologous promoter can depend on a number of factors such as, for example, the desired timing, localization, and pattern of expression as well as responsiveness to a particular biotic or abiotic stimulus. Promoters of interest include, but are not limited to, pathogen-inducible, constitutive, tissue-preferred, wound-inducible, and chemical-regulated promoters.

In certain embodiments of the invention, the transgenic plant, particularly a transgenic wheat plant, can comprise one, two, three, four, five, six, or more nucleotide sequences encoding an R protein. Typically, but not necessarily, the two or more R proteins will be different from each other. For the present invention, an R protein is different from another R protein when the two R proteins have non-identical amino acid sequences. In certain embodiments of the invention, each of the different R proteins for stem rust has one or more differences in resistance characteristics such as, for example, resistance against a different race and/or group of races of Pgt. It is recognized that by combining two, three, four, five, six, or more nucleotide sequences with each nucleotide sequence encoding a different R protein for wheat stem rust, a wheat plant can be produced that comprises broad spectrum resistance against multiple races of Pgt. Such a wheat plant finds use in agriculture in regions where multiple races of Pgt are known to occur.

Examples of wheat stem rust R genes that can be combined in a single wheat plant with a nucleotide sequence of the present invention include Sr22 (WO 2017/024053), Sr26, Sr32, Sr33 (GenBank Accession No. KF031299 1), Sr35 (GenBank Accession No. KC573058.1), Sr39, Sr40, Sr45 (WO 2017/024053), Sr47, Sr50, SrTA1662 (WO 2019140351), and the adult plant resistance gene Sr57/Lr34 (GenBank Accession No. FJ436983.1) and Sr55 Lr67.

A transgenic plant of the invention comprising multiple R genes can be produced by transforming a plant that already comprises one or more other R gene nucleotide sequences with a polynucleotide construct comprising one or more R gene nucleotide sequences of the invention including, for example, an Sr43 nucleotide sequence or variant thereof and/or or an Sr62 nucleotide sequence or variant thereof. Such a plant that already comprises one or more other R gene nucleotide sequences can comprise R genes that are native to the genome of the plant, that were introduced into the plant via sexual reproduction, or that were introduced by transforming the plant or a progenitor thereof with an R gene nucleotide sequence. Alternatively, the one or more other R gene nucleotide sequences can be introduced into a transgenic plant of the invention, which already comprises a polynucleotide construct of the invention, by, for example, transformation or sexual reproduction.

In other embodiments, two or more different R gene sequences can be introduced into a plant by stably transforming the plant with a polynucleotide construct or vector comprising two or more R gene nucleotide sequences. It is recognized that such an approach can be preferred for plant breeding as it is expected that the two or more R gene nucleotide sequences will be tightly linked and thus, segregate as a single locus. Alternatively, a polynucleotide construct of the present invention can be incorporated into the genome of a plant in the immediate vicinity of another R gene nucleotide sequence using homologous recombination-based genome modification methods that are described elsewhere herein or otherwise known in the art.

The present invention further provides methods for enhancing the resistance of a plant, particularly a wheat, barley, or triticale plant, to stem rust caused by Pgt. The methods comprise introducing a polynucleotide construct of the invention into at least one plant cell. In certain embodiments, the polynucleotide construct is stably incorporated into the genome of a plant cell. If desired, the methods can further comprise regenerating the plant cell into a plant comprising in its genome the polynucleotide construct. Preferably, such a regenerated plant comprises enhanced resistance to stem rust caused by at least one race of Pgt, relative to the resistance of a control plant to stem rust caused by the same race or races of Pgt. If desired, the methods can further comprise producing a plant, as described above, comprising one, two, three, four, five, six, or more nucleotide sequences encoding an R protein, preferably each nucleotide sequence encoding a different R protein.

The plants disclosed herein find use in methods for limiting stem rust caused by Pgt in agricultural crop production, particularly in regions where stem rust is prevalent. The methods of the invention comprise planting a seed produced by a plant of the present invention, wherein the seed comprises at least one R gene nucleotide sequence of the present invention. The methods further comprise growing a plant under conditions favorable for the growth and development of the plant therefrom, and optionally harvesting at least one seed, or other plant part or parts, from the plant.

The present invention additionally provides methods for identifying a plant, particularly a wheat, barley, or triticale plant, that displays newly conferred or enhanced resistance to stem rust caused by Pgt. The methods find use in breeding plants for resistance to stem rust. Such resistant plants find use in the agricultural production of wheat seeds. The methods comprise detecting in a plant the presence of at least one R gene of the present invention, particularly Sr43 and/or Sr62. In some embodiments of the invention, detecting the presence of the R gene comprises detecting the entire R gene in genomic DNA isolated from the plant. In preferred embodiments, however, detecting the presence of an R gene comprises detecting the presence of at least one marker within the R gene. In other embodiments of the invention, detecting the presence of an R gene comprises detecting the presence of the R protein encoded by the R gene using, for example, immunological detection methods involving antibodies specific to the R protein.

In the methods for identifying a plant that displays newly conferred or enhanced resistance to stem rust caused by Pgt, detecting the presence of the R gene in the plant can involve one or more of the following molecular biology techniques that are disclosed elsewhere herein or otherwise known in the art including, but not limited to, isolating genomic DNA and/or RNA from the wheat plant, amplifying nucleic acid molecules comprising the R gene and/or marker therein by PCR amplification, sequencing nucleic acid molecules comprising the R gene and/or marker, identifying the R gene, the marker, or a transcript of the R gene by nucleic acid hybridization, and conducting an immunological assay for the detection of the R protein encoded by the R gene. It is recognized that oligonucleotide probes and PCR primers can be designed to identify the R genes of the present invention and that such probes and PCR primers can be utilized in methods disclosed elsewhere herein or otherwise known in the art to rapidly identify in a population of plants one or more plants comprising the presence of an R gene of the present invention. It is further recognized that detecting the presence of the R gene can involve detecting the presence of a fragment of the R gene of the present invention. Such a fragment of an R gene of the present invention can comprise, for example, at least 10, 20, 50, 75, 100, 125, 150, 175, 200, 300, 400, 500, 600, 700, 800, 900, 1000, 1500, 2000, 2500, 3000, 3500, or more contiguous nucleotides.

Depending on the desired outcome, the polynucleotide constructs of the invention can be stably incorporated into the genome of the plant cell or not stably incorporated into the genome of the plant cell. If, for example, the desired outcome is to produce a stably transformed plant with enhanced resistance to wheat stem rust caused by at least one race of Pgt, then the polynucleotide construct can be, for example, fused into a plant transformation vector suitable for the stable incorporation of the polynucleotide construct into the genome of the plant cell. Typically, the stably transformed plant cell will be regenerated into a transformed plant that comprises in its genome the polynucleotide construct. Such a stably transformed plant is capable of transmitting the polynucleotide construct to progeny plants in subsequent generations via sexual and/or asexual reproduction. Plant transformation vectors, methods for stably transforming plants with an introduced polynucleotide construct and methods for plant regeneration from transformed plant cells and tissues are generally known in the art for both monocotyledonous and dicotyledonous plants or described elsewhere herein.

The present invention provides nucleic acid molecules comprising R genes. Preferably, the R genes are capable of conferring upon a host plant, particularly a wheat, barley, or triticale plant, enhanced resistance to at least one race of the pathogen that causes stem rust, Pgt. More preferably, the R genes are capable of conferring upon a host plant, particularly a wheat, barley, or triticale plant, enhanced resistance to two, three, four, or more races of Pgt. Thus, such R genes find use in limiting stem rust caused by Pgt in agricultural production. The R genes of the present invention include, but are not limited to, the R genes whose nucleotide sequences are disclosed herein but also include orthologs and other variants that are capable of conferring to a plant resistance to stem rust caused by at least one race of Pgt. Methods are known in the art or otherwise disclosed herein for determining resistance of a plant to stem rust caused by at least one race of Pgt.

The methods of the present invention find use in producing plants, particularly wheat, barley, and triticale plants, with enhanced resistance to stem rust caused by at least one race of Pgt. Typically, the methods of the present invention will enhance or increase the resistance of the subject plant to one race of Pgt by at least 25%, 50%, 75%, 100%, 150%, 200%, 250%, 500% or more when compared to the resistance of a control plant to the same race or races of Pgt. Unless stated otherwise or apparent from the context of a use, a control plant for the present invention is a plant that does not comprise the polynucleotide construct of the present invention. Preferably, the control plant is essentially identical (e.g. same species, subspecies, and variety) to the plant comprising the polynucleotide construction of the present invention except the control does not comprise the polynucleotide construct. In some embodiments, the control will comprise a polynucleotide construct but not comprise the one or more R gene sequences that are in a polynucleotide construct of the present invention.

Additionally, the present invention provides transformed plants, seeds, and plant cells produced by the methods of present invention and/or comprising a polynucleotide construct of the present invention. Also provided are progeny plants and seeds thereof comprising a polynucleotide construct of the present invention. The present invention also provides seeds, vegetative parts, and other plant parts produced by the transformed plants and/or progeny plants of the invention as well as food products and other agricultural products produced from such plant parts that are intended to be consumed or used by humans and other animals including, but not limited to pets (e.g., dogs and cats) and livestock (e.g., pigs, cows, chickens, turkeys, and ducks).

The methods of the invention can be used to enhance the resistance of a plant, particularly a wheat, barley, or triticale plant, to stem rust, particularly stem rust caused by at least one race of Pgt. As used herein, the term “wheat plant” generally refers to a plant that is a member of the Triticum genus or a member of another genus within the Triticeae tribe, particularly a member of another genus that is capable of producing interspecific hybrids with at least one Triticum sp. Examples of such another genus within the Triticeae tribe are Aegilops and Secale.

The wheat plants of the present invention include, for example, domesticated and non-domesticated plants. The wheat plants of the present invention include, but are not limited to, the following Triticum, Aegilops and Secale species: T. aestivum, T. monococcum, T. turgidum, T. boeoticum, T. timopheevii, and T. urartu, Aegilops tauschii, Secale cereale, and hybrids thereof. Examples of T. aestivum subspecies included within the present invention are aestivum (common wheat), compactum (club wheat), macha (macha wheat), vavilovi (vavilovi wheat), spelta, and sphaecrococcum (shot wheat). Examples of T. turgidum subspecies included within the present invention are turgidum, carthlicum, dicoccom, durum, paleocoichicum, polonicum, turanicum, and dicoccoides. Examples of T. monococcum subspecies included within the present invention are monococcum (einkorn) and aegilopoides. In one embodiment of the present invention, the wheat plant is a member of the Triticum turgidum species; and in particular, a member of the Durum subspecies, for example, a Ciccio, Colosseo, or Utopia cultivar. It is recognized that a wheat plant of the present invention can be a domesticated wheat plant or a non-domesticated wheat plant.

The present invention also encompasses triticale plants, triticale plant parts, and triticale plant cells comprising an R gene of the invention. As used herein, a “triticale plant” refers to a plant that is created by crossing a rye plant (Secale cereale) with either a tetraploid wheat plant (e.g. Triticum turgidum) or a hexaploid wheat plant (e.g. Triticum aestivum). The present invention also includes seeds produced by the triticale plants described herein and methods for controlling weeds in the vicinity of the triticale plants described herein. As used herein, the term “wheat plant” encompasses triticale plants unless stated otherwise or apparent from the context of use.

The term “plant” is intended to encompass plants at any stage of maturity or development, as well as any tissues or organs (plant parts) taken or derived from any such plant unless otherwise clearly indicated by context. As used herein, the term “plant” includes, but is not limited to, seeds, plant cells, plant protoplasts, plant cell tissue cultures from which plants can be regenerated, plant calli, plant clumps, and plant cells that are intact in plants or parts of plants such as embryos, pollen, ovules, seeds, tubers, propagules, leaves, flowers, branches, fruits, roots, root tips, anthers, and the like. The present invention also includes seeds produced by the plants of the present invention.

Progeny, variants, and mutants of the regenerated plants are also included within the scope of the invention, provided that these parts comprise the introduced polynucleotides. As used herein, “progeny” and “progeny plant” comprise any subsequent generation of a plant whether resulting from sexual reproduction and/or asexual propagation, unless it is expressly stated otherwise or is apparent from the context of usage.

Plant parts include, but are not limited to, seeds, stems, roots, flowers, ovules, stamens, leaves, embryos, meristematic regions, callus tissue, anther cultures, gametophytes, sporophytes, pollen, microspores, protoplasts, and the like.

In one embodiment of the invention, the nucleotide sequences encoding R proteins have at least about 80%, 85%, 90%, 91%, 92%, 93%, 94%, 95%, 96%, 97%, 98%, 99% or more sequence identity to the entire nucleotide sequence set forth in SEQ ID NO: 1, 6, 7, 8, 9, 10, 11, 12, 13, 14, 16, or 17, or to a fragment thereof.

The present invention encompasses isolated or substantially purified polynucleotide (also referred to herein as “nucleic acid molecule”, “nucleic acid” and the like) or protein (also referred to herein as “polypeptide”) compositions including, for example, polynucleotides and proteins comprising the sequences set forth in the accompanying Sequence Listing as well as variants and fragments of such polynucleotides and proteins. An “isolated” or “purified” polynucleotide or protein, or biologically active portion thereof, is substantially or essentially free from components that normally accompany or interact with the polynucleotide or protein as found in its naturally occurring environment. Thus, an isolated or purified polynucleotide or protein is substantially free of other cellular material or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various embodiments, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. A protein that is substantially free of cellular material includes preparations of protein having less than about 30%, 20%, 10%, 5%, or 1% (by dry weight) of contaminating protein. When the protein of the invention or biologically active portion thereof is recombinantly produced, optimally culture medium represents less than about 30%, 20%, 10%, 5%, or 1% (by dry weight) of chemical precursors or non-protein-of-interest chemicals.

Fragments and variants of the disclosed polynucleotides and proteins encoded thereby are also encompassed by the present invention. By “fragment” it is intended a portion of the polynucleotide or a portion of the amino acid sequence and hence protein encoded thereby. Fragments of polynucleotides comprising coding sequences may encode protein fragments that retain biological activity of the full-length or native protein. Alternatively, fragments of a polynucleotide that are useful as hybridization probes generally do not encode proteins that retain biological activity or do not retain promoter activity. Thus, fragments of a nucleotide sequence may range from at least about 20 nucleotides, about 50 nucleotides, about 100 nucleotides, and up to the full-length polynucleotide of the invention.

Polynucleotides that are fragments of a native R polynucleotide comprise at least 16, 20, 50, 75, 100, 125, 150, 175, 200, 300, 400, 500, 1000, 1500, 2000, 2500, 3000, or 3500 contiguous nucleotides, or up to the number of nucleotides present in a full-length R polynucleotide disclosed herein.

“Variants” is intended to mean substantially similar sequences. For polynucleotides, a variant comprises a polynucleotide having deletions (i.e., truncations) at the 5′ and/or 3′ end; deletion and/or addition of one or more nucleotides at one or more internal sites in the native polynucleotide; and/or substitution of one or more nucleotides at one or more sites in the native polynucleotide. As used herein, a “native” polynucleotide or polypeptide comprises a naturally occurring nucleotide sequence or amino acid sequence, respectively. For polynucleotides, conservative variants include those sequences that, because of the degeneracy of the genetic code, encode the amino acid sequence of one of the R proteins of the invention. Naturally occurring allelic variants such as these can be identified with the use of well-known molecular biology techniques, as, for example, with polymerase chain reaction (PCR) and hybridization techniques as outlined below. Variant polynucleotides also include synthetically derived polynucleotides, such as those generated, for example, by using site-directed mutagenesis but which still encode an R protein of the invention. Generally, variants of a particular polynucleotide of the invention will have at least about 80%, 85%, 90%, 91%, 92%, 93%, 94%, 95%, 96%, 97%, 98%, 99% or more sequence identity to that particular polynucleotide as determined by sequence alignment programs and parameters as described elsewhere herein. In certain embodiments of the invention, variants of a particular polynucleotide of the invention will have at least about 80%, 85%, 90%, 91%, 92%, 93%, 94%, 95%, 96%, 97%, 98%, 99% or more sequence identity to at least one nucleotide sequence selected from the group consisting of SEQ ID NOS: 1, 6, 7, 8, 9, 10, 11, 12, 13, 14, 16, and 17, and optionally comprises a non-naturally occurring nucleotide sequence that differs from the nucleotide sequence set forth in SEQ ID NO: 1, 6, 7, 8, 9, 10, 11, 12, 13, 14, 16, and/or 17 by at least one nucleotide modification selected from the group consisting of the substitution of at least one nucleotide, the addition of at least one nucleotide, and the deletion of at least one nucleotide.

Variants of a particular polynucleotide of the invention (i.e., the reference polynucleotide) can also be evaluated by comparison of the percent sequence identity between the polypeptide encoded by a variant polynucleotide and the polypeptide encoded by the reference polynucleotide. Thus, for example, a polynucleotide that encodes a polypeptide with a given percent sequence identity to the polypeptide of SEQ ID NO: 2, 3, 4, 5, or 15 is disclosed. Percent sequence identity between any two polypeptides can be calculated using sequence alignment programs and parameters described elsewhere herein. Where any given pair of polynucleotides of the invention is evaluated by comparison of the percent sequence identity shared by the two polypeptides they encode, the percent sequence identity between the two encoded polypeptides is at least about 80%, 85%, 86%, 87%, 88%, 89%, 90%, 91%, 92%, 93%, 94%, 95%, 96%, 97%, 98%, 99% or more sequence identity. In certain embodiments of the invention, variants of a particular polypeptide of the invention will have at least about 80%, 85%, 90%, 91%, 92%, 93%, 94%, 95%, 96%, 97%, 98%, 99% or more sequence identity to the amino acid sequence set forth SEQ ID NO: 2, 3, 4, 5, or 15, and optionally comprises a non-naturally occurring amino acid sequence that differs from the amino acid set forth in SEQ ID NO: 2, 3, 4, 5, or 15 by at least one amino acid modification selected from the group consisting of the substitution of at least one amino acid, the addition of at least one amino acid, and the deletion of at least one amino acid.

“Variant” protein is intended to mean a protein derived from the native protein by deletion (so-called truncation) of one or more amino acids at the N-terminal and/or C-terminal end of the native protein; deletion and/or addition of one or more amino acids at one or more internal sites in the native protein; or substitution of one or more amino acids at one or more sites in the native protein. Such variants may result from, for example, genetic polymorphism or from human manipulation. Biologically active variants of an R protein will have at least about 80%, 85%, 86%, 87%, 88%, 89%, 90%, 91%, 92%, 93%, 94%, 95%, 96%, 97%, 98%, 99% or more sequence identity to the amino acid sequence for the native protein (e.g. the amino acid sequence set forth in SEQ ID NO: 2, 3, 4, 5, or 15) as determined by sequence alignment programs and parameters described elsewhere herein. A biologically active variant of a protein of the invention may differ from that protein by as few as 1-15 amino acid residues, as few as 1-10, such as 6-10, as few as 5, as few as 4, 3, 2, or even 1 amino acid residue.

The proteins of the invention may be altered in various ways including amino acid substitutions, deletions, truncations, and insertions. Methods for such manipulations are generally known in the art. Methods for mutagenesis and polynucleotide alterations are well known in the art. See, for example, Kunkel (1985) PNAS 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be optimal.

Thus, the genes and polynucleotides of the invention include both the naturally occurring sequences as well as mutant and other variant forms. Likewise, the proteins of the invention encompass both naturally occurring proteins as well as variations and modified forms thereof. More preferably, such variants confer to a plant or part thereof comprising the variant enhanced resistance stem rust caused by at least one race of Pgt. In some embodiments, the mutations that will be made in the DNA encoding the variant will not place the sequence out of reading frame. Optimally, the mutations will not create complementary regions that could produce secondary mRNA structure. See, EP Patent Application Publication No. 75,444.

The deletions, insertions, and substitutions of the protein sequences encompassed herein are not expected to produce radical changes in the characteristics of the protein. However, when it is difficult to predict the exact effect of the substitution, deletion, or insertion in advance of doing so, one skilled in the art will appreciate that the effect will be evaluated by routine screening assays. That is, the activity can be evaluated by assays that are disclosed herein below.

For example, a wheat plant that is susceptible to wheat stem rust caused by a particular race of Pgt can be transformed with a polynucleotide comprising an Sr43 nucleotide sequence and/or an Sr62 nucleotide sequence, regenerated into a transformed or transgenic plant comprising the polynucleotide, and tested for resistance to wheat stem rust caused by the particular race of Pgt using standard resistance assays known in the art or described elsewhere herein. Preferred variant polynucleotides and polypeptides of the present invention confer or are capable of conferring upon a wheat plant enhanced resistance to at least one race of Pgt that is known to cause wheat stem rust in a susceptible wheat plant.

Variant polynucleotides and proteins also encompass sequences and proteins derived from a mutagenic and recombinogenic procedure such as DNA shuffling. Strategies for such DNA shuffling are known in the art. See, for example, Stemmer (1994) PNAS 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) PNAS 94:4504-4509; Crameri et al. (1998) Nature 391:288-291; and U.S. Pat. Nos. 5,605,793 and 5,837,458.

The polynucleotides of the invention can be used to isolate corresponding sequences from other organisms, particularly other plants. In this manner, methods such as PCR, hybridization, and the like can be used to identify such sequences based on their sequence homology to the sequences set forth herein. Sequences isolated based on their sequence identity to the entire sequences set forth herein or to variants and fragments thereof are encompassed by the present invention. Such sequences include sequences that are orthologs of the disclosed sequences. “Orthologs” is intended to mean genes derived from a common ancestral gene and which are found in different species as a result of speciation. Genes found in different species are considered orthologs when their nucleotide sequences and/or their encoded protein sequences share at least 80%, 85%, 86%, 87%, 88%, 89%, 90%, 91%, 92%, 93%, 94%, 95%, 96%, 97%, 98%, 99%, or greater sequence identity. Functions of orthologs are often highly conserved among species. Thus, isolated polynucleotides that encode R proteins and which hybridize under stringent conditions to at least one of the R proteins disclosed herein or otherwise known in the art, or to variants or fragments thereof, are encompassed by the present invention.

In one embodiment, the orthologs of the present invention have coding sequences comprising at least 80%, 85%, 86%, 87%, 88%, 89%, 90%, 91%, 92%, 93%, 94%, 95%, 96%, 97%, 98%, 99%, or greater nucleotide sequence identity to a nucleotide sequence selected from the group consisting of the nucleotide sequences set forth in SEQ ID NOS: 1, 6, 7, 8, 9, 10, 11, 12, 13, 14, 16, and 17, and/or encode proteins comprising at least 80%, 85%, 86%, 87%, 88%, 89%, 90%, 91%, 92%, 93%, 94%, 95%, 96%, 97%, 98%, 99%, or greater amino acid sequence identity to the amino acid sequence set forth in SEQ ID NO: 2, 3, 4, 5, or 15.

Preferably, the variant Sr43 and Sr62 proteins of the present invention and the polynucleotides encoding them confer, or are capable of conferring upon a wheat plant comprising such a protein and/or polynucleotide, enhanced resistance to at least one race of Pgt that is known to cause wheat stem rust in a susceptible wheat plant.

In a PCR approach, oligonucleotide primers can be designed for use in PCR reactions to amplify corresponding DNA sequences from cDNA or genomic DNA extracted from any plant of interest. Methods for designing PCR primers and PCR cloning are generally known in the art and are disclosed in Sambrook et al. (1989) Molecular Cloning: A Laboratory Manual (2d ed., Cold Spring Harbor Laboratory Press, Plainview, New York). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially-mismatched primers, and the like.

It is recognized that the R protein coding sequences of the present invention encompass polynucleotide molecules comprising a nucleotide sequence that is sufficiently identical to the nucleotide sequence of any one or more of SEQ ID NOS: 1, 2, and 4. The term “sufficiently identical” is used herein to refer to a first amino acid or nucleotide sequence that contains a sufficient or minimum number of identical or equivalent (e.g., with a similar side chain) amino acid residues or nucleotides to a second amino acid or nucleotide sequence such that the first and second amino acid or nucleotide sequences have a common structural domain and/or common functional activity. For example, amino acid or nucleotide sequences that contain a common structural domain having at least about 80% or 85% identity, preferably 90% or 91% identity, more preferably 92%, 93%, 94%, 95%, 96%, 97%, 98% or 99% identity are defined herein as sufficiently identical.

To determine the percent identity of two amino acid sequences or of two nucleic acids, the sequences are aligned for optimal comparison purposes. The percent identity between the two sequences is a function of the number of identical positions shared by the sequences (i.e., percent identity=number of identical positions/total number of positions (e.g., overlapping positions)×100). In one embodiment, the two sequences are the same length. The percent identity between two sequences can be determined using techniques similar to those described below, with or without allowing gaps. In calculating percent identity, typically exact matches are counted.

The determination of percent identity between two sequences can be accomplished using a mathematical algorithm. A preferred, nonlimiting example of a mathematical algorithm utilized for the comparison of two sequences is the algorithm of Karlin and Altschul (1990) PNAS 87:2264, modified as in Karlin and Altschul (1993) PNAS 90:5873-5877. Such an algorithm is incorporated into the NBLAST and XBLAST programs of Altschul et al. (1990) J. Mol. Biol. 215:403. BLAST nucleotide searches can be performed with the NBLAST program, score=100, wordlength=12, to obtain nucleotide sequences homologous to the polynucleotide molecules of the invention. BLAST protein searches can be performed with the XBLAST program, score=50, wordlength=3, to obtain amino acid sequences homologous to protein molecules of the invention. To obtain gapped alignments for comparison purposes, Gapped BLAST can be utilized as described in Altschul et al. (1997) Nucleic Acids Res. 25:3389. Alternatively, PSI-Blast can be used to perform an iterated search that detects distant relationships between molecules. See Altschul et al. (1997) supra. When utilizing BLAST, Gapped BLAST, and PSI-Blast programs, the default parameters of the respective programs (e.g., XBLAST and NBLAST; available on the world-wide web at ncbi.nlm.nih.gov). Another preferred, non-limiting example of a mathematical algorithm utilized for the comparison of sequences is the algorithm of Myers and Miller (1988) CABIOS 4:11-17. Such an algorithm is incorporated into the ALIGN program (version 2.0), which is part of the GCG sequence alignment software package. When utilizing the ALIGN program for comparing amino acid sequences, a PAM120 weight residue table, a gap length penalty of 12, and a gap penalty of 4 can be used. Alignment may also be performed manually by inspection.

Unless otherwise stated, sequence identity/similarity values provided herein refer to the value obtained using the full-length sequences of the invention and using multiple alignment by mean of the algorithm Clustal W (Nucleic Acid Research, 22 (22):4673-4680, 1994) using the program AlignX included in the software package Vector NTI Suite Version 7 (InforMax, Inc., Bethesda, MD, USA) using the default parameters; or any equivalent program thereof. By “equivalent program”, it is intended any sequence comparison program that, for any two sequences in question, generates an alignment having identical nucleotide or amino acid residue matches and an identical percent sequence identity when compared to the corresponding alignment generated by CLUSTALW (Version 1.83) using default parameters (available at the European Bioinformatics Institute website on the world-wide web at: ebi.ac.uk/Tools/clustalw/index.html).

The use of the term “polynucleotide” is not intended to limit the present invention to polynucleotides comprising DNA. Those of ordinary skill in the art will recognize that polynucleotides, can comprise ribonucleotides and combinations of ribonucleotides and deoxyribonucleotides. Such deoxyribonucleotides and ribonucleotides include both naturally occurring molecules and synthetic analogues. The polynucleotides of the invention also encompass all forms of sequences including, but not limited to, single-stranded forms, double-stranded forms, hairpins, stem-and-loop structures, and the like.

The polynucleotide constructs comprising R protein coding regions can be provided in expression cassettes for expression in the plant or other organism or non-human host cell of interest. The cassette will include 5′ and 3′ regulatory sequences operably linked to the R protein coding region. “Operably linked” is intended to mean a functional linkage between two or more elements. For example, an operable linkage between a polynucleotide or gene of interest and a regulatory sequence (i.e., a promoter) is a functional link that allows for expression of the polynucleotide of interest. Operably linked elements may be contiguous or non-contiguous. When used to refer to the joining of two protein coding regions, by operably linked is intended that the coding regions are in the same reading frame. The cassette may additionally contain at least one additional gene to be cotransformed into the organism. Alternatively, the additional gene(s) can be provided on multiple expression cassettes. Such an expression cassette is provided with a plurality of restriction sites and/or recombination sites for insertion of the R protein coding region to be under the transcriptional regulation of the regulatory regions. The expression cassette may additionally contain selectable marker genes.

The expression cassette will include in the 5′-3′ direction of transcription, a transcriptional and translational initiation region (i.e., a promoter), a R protein coding region of the invention, and a transcriptional and translational termination region (i.e., termination region) functional in plants or other organism or non-human host cell. The regulatory regions (i.e., promoters, transcriptional regulatory regions, and translational termination regions) and/or the R protein coding region of the invention may be native/analogous to the host cell or to each other. Alternatively, the regulatory regions and/or the R protein coding region of the invention may be heterologous to the host cell or to each other.

As used herein, “heterologous” in reference to a nucleic acid molecule or nucleotide sequence is a nucleic acid molecule or nucleotide sequence that originates from a foreign species, or, if from the same species, is modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide. As used herein, a chimeric gene comprises a coding sequence operably linked to a transcription initiation region that is heterologous to the coding sequence.

The present invention provides host cells comprising at least one of the nucleic acid molecules, expression cassettes, and vectors of the present invention. In preferred embodiments of the invention, a host cell is a plant cell. In other embodiments, a host cell is selected from the group consisting of a bacterium, a fungal cell, and an animal cell. In certain embodiments, a host cell is a non-human animal cell. However, in some other embodiments, the host cell is an in-vitro cultured human cell. In yet some other embodiments, the host cell is a microorganism, particularly a unicellular microorganism. Microorganisms include, but are not limited to, archaebacteria, eubacteria, yeasts, and algae.

While it may be optimal to express the R protein using heterologous promoters, the native promoter of the corresponding R gene may be used.

The termination region may be native with the transcriptional initiation region, may be native with the operably linked R protein coding region of interest, may be native with the plant host, or may be derived from another source (i.e., foreign or heterologous to the promoter, the R protein of interest, and/or the plant host), or any combination thereof. Convenient termination regions are available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nuc. Acids Res. 17:7891-7903; and Joshi et al. (1987) Nucleic Acids Res. 15:9627-9639.

Where appropriate, the polynucleotides may be optimized for increased expression in the transformed plant. That is, the polynucleotides can be synthesized using plant-preferred codons for improved expression. See, for example, Campbell and Gowri (1990) Plant Physiol. 92:1-11 for a discussion of host-preferred codon usage. Methods are available in the art for synthesizing plant-preferred genes. See, for example, U.S. Pat. Nos. 5,380,831, and 5,436,391, and Murray et al. (1989) Nucleic Acids Res. 17:477-498, herein incorporated by reference.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The expression cassettes may additionally contain 5′ leader sequences. Such leader sequences can act to enhance translation. Translation leaders are known in the art and include: picornavirus leaders, for example, EMCV leader (Encephalomyocarditis 5′ noncoding region) (Elroy-Stein et al. (1989) PNAS 86:6126-6130); potyvirus leaders, for example, TEV leader (Tobacco Etch Virus) (Gallie et al. (1995) Gene 165 (2):233-238), MDMV leader (Maize Dwarf Mosaic Virus) (Virology 154:9-20), and human immunoglobulin heavy-chain binding protein (BiP) (Macejak et al. (1991) Nature 353:90-94); untranslated leader from the coat protein mRNA of alfalfa mosaic virus (AMV RNA 4) (Jobling et al. (1987) Nature 325:622-625); tobacco mosaic virus leader (TMV) (Gallie et al. (1989) in Molecular Biology of RNA, ed. Cech (Liss, New York), pp. 237-256); and maize chlorotic mottle virus leader (MCMV) (Lommel et al. (1991) Virology 81:382-385). See also, Della-Cioppa et al. (1987) Plant Physiol. 84:965-968.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

A number of promoters can be used in the practice of the invention. The promoters can be selected based on the desired outcome. The nucleic acids can be combined with constitutive, tissue-preferred, or other promoters for expression in plants. Such constitutive promoters include, for example, the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142; and 6,177,611.

Tissue-preferred promoters can be utilized to target enhanced expression of the R protein coding sequences within a particular plant tissue. Such tissue-preferred promoters include, but are not limited to, leaf-preferred promoters, root-preferred promoters, seed-preferred promoters, and stem-preferred promoters. Tissue-preferred promoters include Yamamoto et al. (1997) Plant J. 12 (2):255-265; Kawamata et al. (1997) Plant Cell Physiol. 38 (7):792-803; Hansen et al. (1997) Mol. Gen Genet. 254 (3):337-343; Russell et al. (1997) Transgenic Res. 6 (2):157-168; Rinehart et al. (1996) Plant Physiol. 112 (3):1331-1341; Van Camp et al. (1996) Plant Physiol. 112 (2):525-535; Canevascini et al. (1996) Plant Physiol. 112 (2):513-524; Yamamoto et al. (1994) Plant Cell Physiol. 35 (5):773-778; Lam (1994) Results Probl. Cell Differ. 20:181-196; Orozco et al. (1993) Plant Mol Biol. 23 (6):1129-1138; Matsuoka et al. (1993) PNAS 90 (20):9586-9590; and Guevara-Garcia et al. (1993) Plant J. 4 (3):495-505. Such promoters can be modified, if necessary, for weak expression.

Generally, it will be beneficial to express the gene from an inducible promoter, particularly from a pathogen-inducible promoter. Such promoters include those from pathogenesis-related proteins (PR proteins), which are induced following infection by a pathogen; e.g., PR proteins, SAR proteins, beta-1,3-glucanase, chitinase, etc. See, for example, Redolfi et al. (1983) Neth. J. Plant Pathol. 89:245-254; Uknes et al. (1992) Plant Cell 4:645-656; and Van Loon (1985) Plant Mol. Virol. 4:111-116. See also WO 99/43819, herein incorporated by reference.

Of interest are promoters that are expressed locally at or near the site of pathogen infection. See, for example, Marineau et al. (1987) Plant Mol. Biol. 9:335-342; Matton et al. (1989) Molecular Plant-Microbe Interactions 2:325-331; Somsisch et al. (1986) PNAS 83:2427-2430; Somsisch et al. (1988) Mol. Gen. Genet. 2:93-98; and Yang (1996) PNAS 93:14972-14977. See also, Chen et al. (1996) Plant J. 10:955-966; Zhang et al. (1994) PNAS 91:2507-2511; Warner et al. (1993) Plant J. 3:191-201; Siebertz et al. (1989) Plant Cell 1:961-968; U.S. U.S. Pat. No. 5,750,386 (nematode-inducible); and the references cited therein. Of particular interest is the inducible promoter for the maize PRms gene, whose expression is induced by the pathogen Fusarium moniliforme (see, for example, Cordero et al. (1992) Physiol. Mol. Plant Path. 41:189-200).

Also of interest are the native promoters from other resistance genes from the target species. These promoters are often pathogen-inducible, and are likely to express the resistance gene at appropriate levels and in appropriate tissues. Examples of such promoters are the Sr57/Lr34, Sr33, Sr35, and Sr22 promoters of wheat (Risk et al. (2012) Plant Biotechnol J 10:447-487; Periyannan et al. (2013) Science 341:786-788; Steuernagel et al. (2016) Nature Biotechnol. 34 (6):652-655, doi: 10.1038/nbt.3543; Hatta et al. (2020) Plant Biotechnol. J. 19 (2): 273-284; Hatta et al. (2020) Mol. Plant Microbe Interact. 33 (11):1286-1298).

Additionally, as pathogens find entry into plants through wounds or insect damage, a wound-inducible promoter may be used in the constructions of the invention. Such wound-inducible promoters include potato proteinase inhibitor (pin II) gene (Ryan (1990) Ann. Rev. Phytopath. 28:425-449; Duan et al. (1996) Nature Biotechnology 14:494-498); wun1 and wun2, U.S. Patent No. 5,428, 148; win1 and win2 (Stanford et al. (1989) Mol. Gen. Genet. 215:200-208); systemin (McGurl et al. (1992) Science 225:1570-1573); WIP1 (Rohmeier et al. (1993) Plant Mol. Biol. 22:783-792; Eckelkamp et al. (1993) FEBS Letters 323:73-76); MPI gene (Corderok et al. (1994) Plant J. 6 (2):141-150); and the like, herein incorporated by reference.

Chemical-regulated promoters can be used to modulate the expression of a gene in a plant through the application of an exogenous chemical regulator. Depending upon the objective, the promoter may be a chemical-inducible promoter, where application of the chemical induces gene expression, or a chemical-repressible promoter, where application of the chemical represses gene expression. Chemical-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-1a promoter, which is activated by salicylic acid. Other chemical-regulated promoters of interest include steroid-responsive promoters (see, for example, the glucocorticoid-inducible promoter in Schena et al. (1991) PNAS 88:10421-10425 and McNellis et al. (1998) Plant J. 14 (2):247-257) and tetracycline-inducible and tetracycline-repressible promoters (see, for example, Gatz et al. (1991) Mol. Gen. Genet. 227:229-237, and U.S. Pat. Nos. 5,814,618 and 5,789,156), herein incorporated by reference.

The expression cassette can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D). Additional selectable markers include phenotypic markers such as β-galactosidase and fluorescent proteins such as green fluorescent protein (GFP) (Su et al. (2004) Biotechnol Bioeng 85:610-9 and Fetter et al. (2004) Plant Cell 16:215-28), cyan florescent protein (CYP) (Bolte et al. (2004) J. Cell Science 117:943-54 and Kato et al. (2002) Plant Physiol 129:913-42), and yellow florescent protein (PhiYFP™ from Evrogen, see, Bolte et al. (2004) J. Cell Science 117:943-54). For additional selectable markers, see generally, Yarranton (1992) Curr. Opin. Biotech. 3:506-511; Christopherson et al. (1992) PNAS 89:6314-6318; Yao et al. (1992) Cell 71:63-72; Reznikoff (1992) Mol. Microbiol. 6:2419-2422; Barkley et al. (1980) in The Operon, pp. 177-220; Hu et al. (1987) Cell 48:555-566; Brown et al. (1987) Cell 49:603-612; Figge et al. (1988) Cell 52:713-722; Deuschle et al. (1989) PNAS 86:5400-5404; Fuerst et al. (1989) PNAS 86:2549-2553; Deuschle et al. (1990) Science 248:480-483; Gossen (1993) Ph.D. Thesis, University of Heidelberg; Reines et al. (1993) PNAS 90:1917-1921; Labow et al. (1990) Mol. Cell. Biol. 10:3343-3356; Zambretti et al. (1992) PNAS 89:3952-3956; Baim et al. (1991) PNAS 88:5072-5076; Wyborski et al. (1991) Nucleic Acids Res. 19:4647-4653; Hillenand-Wissman (1989) Topics Mol. Struc. Biol. 10:143-162; Degenkolb et al. (1991) Antimicrob. Agents Chemother. 35:1591-1595; Kleinschmidt et al. (1988) Biochemistry 27:1094-1104; Bonin (1993) Ph.D. Thesis, University of Heidelberg; Gossen et al. (1992) PNAS 89:5547-5551; Oliva et al. (1992) Antimicrob. Agents Chemother. 36:913-919; Hlavka et al. (1985) Handbook of Experimental Pharmacology, Vol. 78 (Springer-Verlag, Berlin); Gill et al. (1988) Nature 334:721-724. Such disclosures are herein incorporated by reference.

The above list of selectable marker genes is not intended to be limiting. Any selectable marker gene can be used in the present invention.

Numerous plant transformation vectors and methods for transforming plants are available. See, for example, An et al. (1986) Plant Physiol., 81:301-305; Fry et al. (1987) Plant Cell Rep. 6:321-325; Block (1988) Theor. Appl. Genet. 76:767-774; Hinchee et al. (1990) Stadler Genet. Symp. 203212.203-212; Cousins, et al. (1991) Aust. J. Plant Physiol. 18:481-494; Chee and Slightom (1992) Gene. 118:255-260; Christou et al. (1992) Trends Biotechnol. 10:239-246; D'Halluin et al. (1992) Bio/Technol. 10:309-314; Dhir et al. (1992) Plant Physiol. 99:81-88; Casas et al. (1993) PNAS 90:11212-11216; Christou (1993) In Vitro Cell. Dev. Biol.-Plant; 29P:119-124; Davies et al. (1993) Plant Cell Rep. 12:180-183; Dongand Mchughen (1993) Plant Sci. 91:139-148; Franklin et al. (1993) Plant Cell Rep. 12 (2):74-79, doi: 10.1007/BF00241938; Golovkin et al. (1993) Plant Sci. 90:41-52; Asano et al. (1994) Plant Cell Rep. 13; Ayeres and Park (1994) Crit. Rev. Plant Sci. 13:219-239; Barcelo et al. (1994) Plant J. 5:583-592; Becker et al. (1994) Plant J. 5:299-307; Borkowska et al. (1994) Acta Physiol. Plant 16:225-230; Christou (1994) Agro. Food Ind. Hi Tech. 5:17-27; Eapen et al. (1994) Plant Cell Rep. 13:582-586; Hartman, et al. (1994) Bio-Technology 12:919923; Ritala et al. (1994) Plant Mol. Biol. 24:317-325; Wan and Lemaux (1994) Plant Physiol. 104:3748; Debernardi et al. (2020) Nat Biotechnol. 38 (11):1274-1279; and Lowe et al. (2016) Plant Cell 28 (9):1998-2015.

The methods of the invention involve introducing a polynucleotide construct into a plant. By “introducing” is intended presenting to the plant the polynucleotide construct in such a manner that the construct gains access to the interior of a cell of the plant. The methods of the invention do not depend on a particular method for introducing a polynucleotide construct to a plant, only that the polynucleotide construct gains access to the interior of at least one cell of the plant. Methods for introducing polynucleotide constructs into plants are known in the art including, but not limited to, stable transformation methods, transient transformation methods, and virus-mediated methods.

By “stable transformation” is intended that the polynucleotide construct introduced into a plant integrates into the genome of the plant and is capable of being inherited by progeny thereof. By “transient transformation” is intended that a polynucleotide construct introduced into a plant does not integrate into the genome of the plant.

For the transformation of plants and plant cells, the nucleotide sequences of the invention are inserted using standard techniques into any vector known in the art that is suitable for expression of the nucleotide sequences in a plant or plant cell. The selection of the vector depends on the preferred transformation technique and the target plant species to be transformed.

Methodologies for constructing plant expression cassettes and introducing foreign nucleic acids into plants are generally known in the art and have been previously described. For example, foreign DNA can be introduced into plants, using tumor-inducing (Ti) plasmid vectors. Other methods utilized for foreign DNA delivery involve the use of PEG mediated protoplast transformation, electroporation, microinjection whiskers, and biolistics or microprojectile bombardment for direct DNA uptake. Such methods are known in the art. (U.S. Pat. No. 5,405,765 to Vasil et al.; Bilang et al. (1991) Gene 100:247-250; Scheid et al., (1991) Mol. Gen. Genet., 228:104-112; Guerche et al., (1987) Plant Science 52:111-116; Neuhause et al., (1987) Theor. Appl Genet. 75:30-36; Klein et al., (1987) Nature 327:70-73; Howell et al., (1980) Science 208:1265; Horsch et al., (1985) Science 227:1229-1231; DeBlock et al., (1989) Plant Physiology 91:694-701; Methods for Plant Molecular Biology (Weissbach and Weissbach, eds.) Academic Press, Inc. (1988) and Methods in Plant Molecular Biology (Schuler and Zielinski, eds.) Academic Press, Inc. (1989). The method of transformation depends upon the plant cell to be transformed, stability of vectors used, expression level of gene products and other parameters.

Other suitable methods of introducing nucleotide sequences into plant cells and subsequent insertion into the plant genome include microinjection as Crossway et al. (1986) Biotechniques 4:320-334, electroporation as described by Riggs et al. (1986) PNAS 83:5602-5606, Agrobacterium-mediated transformation as described by Townsend et al., U.S. Pat. No. 5,563,055, Zhao et al., U.S. Pat. No. 5,981,840, direct gene transfer as described by Paszkowski et al. (1984) EMBO J. 3:2717-2722, and ballistic particle acceleration as described in, for example, Sanford et al., U.S. Pat. No. 4,945,050; Tomes et al., U.S. Patent No. 5,879,918; Tomes et al., U.S. Pat. No. 5,886,244; Bidney et al., U.S. Pat. No. 5,932,782; Tomes et al. (1995) “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin); McCabe et al. (1988) Biotechnology 6:923-926); and Lec1 transformation (WO 00/28058). Also see, Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P: 175-182 (soybean); Singh et al. (1998) Theor. Appl. Genet. 96:319-324 (soybean); Datta et al. (1990) Biotechnology 8:736-740 (rice); Klein et al. (1988) PNAS 85:4305-4309 (maize); Klein et al. (1988) Biotechnology 6:559-563 (maize); Tomes, U.S. Pat. No. 5,240,855; Buising et al., U.S. Pat. Nos. 5,322,783 and 5,324,646; Tomes et al. (1995) “Direct DNA Transfer into Intact Plant Cells via Microprojectile Bombardment,” in Plant Cell, Tissue, and Organ Culture: Fundamental Methods, ed. Gamborg (Springer-Verlag, Berlin) (maize); Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; Bowen et al., U.S. Pat. No. 5,736,369 (cereals); Bytebier et al. (1987) PNAS 84:5345-5349 (Liliaceae); De Wet et al. (1985) in The Experimental Manipulation of Ovule Tissues, ed. Chapman et al. (Longman, New York), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

The polynucleotides of the invention may be introduced into plants by contacting plants with a virus or viral nucleic acids. Generally, such methods involve incorporating a polynucleotide construct of the invention within a viral DNA or RNA molecule. Further, it is recognized that promoters of the invention also encompass promoters utilized for transcription by viral RNA polymerases. Methods for introducing polynucleotide constructs into plants and expressing a protein encoded therein, involving viral DNA or RNA molecules, are known in the art. See, for example, U.S. Pat. Nos. 5,889,191, 5,889,190, 5,866, 785, 5,589,367 and 5,316,931; herein incorporated by reference.

If desired, the modified viruses or modified viral nucleic acids can be prepared in formulations. Such formulations are prepared in a known manner (see e.g. for review U.S. Pat. No. 3,060,084, EP-A 707 445 (for liquid concentrates), Browning, “Agglomeration”, Chemical Engineering, Dec. 4, 1967, 147-48, Perry's Chemical Engineer's Handbook, 4th Ed., McGraw-Hill, New York, 1963, pages 8-57 and et seq. WO 91/13546, U.S. Pat. Nos. 4,172,714, 4,144,050, 3,920,442, 5,180,587, 5,232,701, 5,208,030, GB 2,095,558, U.S. Pat. No. 3,299,566, Klingman, Weed Control as a Science, John Wiley and Sons, Inc., New York, 1961, Hance et al. Weed Control Handbook, 8th Ed., Blackwell Scientific Publications, Oxford, 1989 and Mollet, H., Grubemann, A., Formulation technology, Wiley VCH Verlag GmbH, Weinheim (Germany), 2001, 2. D. A. Knowles, Chemistry and Technology of Agrochemical Formulations, Kluwer Academic Publishers, Dordrecht, 1998 (ISBN 0-7514-0443-8), for example by extending the active compound with auxiliaries suitable for the formulation of agrochemicals, such as solvents and/or carriers, if desired emulsifiers, surfactants and dispersants, preservatives, antifoaming agents, anti-freezing agents, for seed treatment formulation also optionally colorants and/or binders and/or gelling agents.

In specific embodiments, the polynucleotide constructs and expression cassettes of the invention can be provided to a plant using a variety of transient transformation methods known in the art. Such methods include, for example, microinjection or particle bombardment. See, for example, Crossway et al. (1986) Mol Gen. Genet. 202:179-185; Nomura et al. (1986) Plant Sci. 44:53-58; Hepler et al. (1994) PNAS Sci. 91:2176-2180 and Hush et al. (1994) J. Cell Science 107:775-784, all of which are herein incorporated by reference. Alternatively, the polynucleotide can be transiently transformed into the plant using techniques known in the art. Such techniques include viral vector system and Agrobacterium tumefaciens-mediated transient expression as described elsewhere herein.

The cells that have been transformed may be grown into plants in accordance with conventional ways. See, for example, McCormick et al. (1986) Plant Cell Reports 5:81-84. These plants may then be grown, and either pollinated with the same transformed line or different lines, and the resulting hybrid having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present invention provides transformed seed (also referred to as “transgenic seed”) having a polynucleotide construct of the invention, for example, an expression cassette of the invention, stably incorporated into their genome.

Any methods known in the art for modifying DNA in the genome of a plant can be used to modify the nucleotide sequences of an R gene in planta, e.g. to modify the nucleotide sequence of a non-functional allele to that of a functional allele that provides resistance to a plant pathogen. Such modifications to the DNA in the genome of a plant include, for example, insertions, deletions, substitutions, and combinations thereof. The insertions, deletions, and substitutions can be made using any method known in the art such as, for example, by genome editing techniques as described elsewhere herein or otherwise known in the art.

The insertions comprise an insertion of at least one nucleotide or base pair (bp) in an allele of an R gene of the present invention. The insertion can comprise insertion of any size DNA fragment into the genome. The inserted DNA can be 1 bp in length, 1-5 bp in length, 5-10 bp in length, 10-15 bp in length, 15-20 bp in length, 20-30 bp in length, 30-50 bp in length, 50-100 bp in length, 100-200 bp in length, 200-300 bp in length, 300-400 bp in length, 400-500 bp in length, 500-600 bp in length, 600-700 bp in length, 700-800 bp in length, 800-900 bp in length, 900-1000 bp in length, 1000-1500 bp.

The deletions comprise the deletion of at least one bp from an allele of an R gene of the present invention. As used herein, a “deletion” is meant the removal of one or more nucleotides or base pairs from the DNA. Provided herein, a deletion in an allele of an R gene can be the removal of at least 1, at least 20, at least 50, at least 100, at least 500, at least 1000, at least 5000 or more bp.

The substitutions comprise the replacement of at least one bp from an allele of an R gene of the present invention with another bp. As used herein, a “substitution” is meant the replacement of one or more nucleotides or base pairs from the DNA with non-identical nucleotides or base pairs. When the substitution comprises two or more nucleotides, the two or more nucleotides can be contiguous or non-contiguous within the DNA sequence of the allele. Provided herein, a substitution in an allele of an R gene can be the replacement of at least 1, at least 20, at least 50, at least 100, at least 500, at least 1000, at least 5000 or more base pairs. In some embodiments, the substitution can be the nucleotide sequence of the entire allele or any portion or portions thereof such as, for example, the transcribed region, the 5′ untranslated region, the 3′ untranslated region, an exon, or an intron.

Any methods known in the art for modifying DNA in the genome of a plant can be used to alter the coding sequences of an R gene in planta. Such methods known in the art for modifying DNA in the genome of a plant include, for example, mutation breeding and genome editing techniques, such as, for example, methods involving targeted mutagenesis, site-directed integration (SDI), and homologous recombination. Targeted mutagenesis or similar techniques are disclosed in U.S. Pat. Nos. 5,565,350; 5,731,181; 5,756,325; 5,760,012; 5,795,972, 5,871,984, and 8,106,259; all of which are herein incorporated in their entirety by reference. Methods for gene modification or gene replacement comprising homologous recombination can involve inducing single-strand or double-strand breaks in DNA using zinc-finger nucleases (ZFN), TAL (transcription activator-like) effector nucleases (TALEN), Clustered Regularly Interspaced Short Palindromic Repeats/CRISPR-associated nuclease (CRISPR/Cas nuclease), or homing endonucleases that have been engineered endonucleases to make double-strand breaks at specific recognition sequences in the genome of a plant, other organism, or host cell. See, for example, Durai et al., (2005) Nucleic Acids Res 33:5978-90; Mani et al. (2005) Biochem Biophys Res Comm 335:447-57; U.S. Pat. Nos. 7,163,824, 7,001,768, and 6,453,242; Arnould et al. (2006) J Mol Biol 355:443-58; Ashworth et al., (2006) Nature 441:656-9; Doyon et al. (2006) J Am Chem Soc 128:2477-84; Rosen et al., (2006) Nucleic Acids Res 34:4791-800; and Smith et al., (2006) Nucleic Acids Res 34:e149; U.S. Pat. App. Pub. No. 2009/0133152; and U.S. Pat. App. Pub. No. 2007/0117128; all of which are herein incorporated in their entirety by reference.

TAL effector nucleases (TALENs) can be used to make double-strand breaks at specific recognition sequences in the genome of a plant for gene modification or gene replacement through homologous recombination. TAL effector nucleases are a class of sequence-specific nucleases that can be used to make double-strand breaks at specific target sequences in the genome of a plant or other organism. TAL effector nucleases are created by fusing a native or engineered transcription activator-like (TAL) effector, or functional part thereof, to the catalytic domain of an endonuclease, such as, for example, FokI. The unique, modular TAL effector DNA binding domain allows for the design of proteins with potentially any given DNA recognition specificity. Thus, the DNA binding domains of the TAL effector nucleases can be engineered to recognize specific DNA target sites and thus, used to make double-strand breaks at desired target sequences. See, WO 2010/079430; Morbitzer et al. (2010) PNAS 10.1073/pnas.1013133107; Scholze and Boch (2010) Virulence 1:428-432; Christian et al. Genetics (2010) 186:757-761; Li et al. (2010) Nuc. Acids Res. (2010) doi:10.1093/nar/gkq704; and Miller et al. (2011) Nature Biotechnology 29:143-148; all of which are herein incorporated by reference.

The CRISPR/Cas nuclease system can also be used to make single-strand or double-strand breaks at specific recognition sequences in the genome of a plant for gene modification or gene replacement through homologous recombination. The CRISPR/Cas nuclease is an RNA-guided (simple guide RNA, sgRNA in short) DNA endonuclease system performing sequence-specific double-stranded breaks in a DNA segment homologous to the designed RNA. It is possible to design the specificity of the sequence (Cho et al. (2013) Nat. Biotechnol. 31:230-232; Cong et al. (2013) Science 339:819-823; Mali et al. (2013) Science 339:823-826; Feng et al. (2013) Cell Res. 23 (10):1229-1232).

In addition, a ZFN can be used to make double-strand breaks at specific recognition sequences in the genome of a plant for gene modification or gene replacement through homologous recombination. The Zinc Finger Nuclease (ZFN) is a fusion protein comprising the part of the FokI restriction endonuclease protein responsible for DNA cleavage and a zinc finger protein which recognizes specific, designed genomic sequences and cleaves the double-stranded DNA at those sequences, thereby producing free DNA ends (Urnov F. D. et al., Nat Rev Genet. 11:636-46, 2010; Carroll D., Genetics. 188:773-82, 2011).

Breaking DNA using site specific nucleases, such as, for example, those described herein above, can increase the rate of homologous recombination in the region of the breakage. Thus, coupling of such effectors as described above with nucleases enables the generation of targeted changes in genomes which include additions, deletions, substitutions, and other modifications.

Mutation breeding can also be used in the methods provided herein. Mutation breeding methods can involve, for example, exposing the plants or seeds to a mutagen, particularly a chemical mutagen such as, for example, ethyl methanesulfonate (EMS) and selecting for plants that possess a desired modification in the R gene of interest. However, other mutagens can be used in the methods disclosed herein including, but not limited to, radiation, such as X-rays, Gamma rays (e.g., cobalt 60 or cesium 137), neutrons, (e.g., product of nuclear fission by uranium 235 in an atomic reactor), Beta radiation (e.g., emitted from radioisotopes such as phosphorus 32 or carbon 14), and ultraviolet radiation (preferably from 2500 to 2900 nm), and chemical mutagens such as base analogues (e.g., 5-bromo-uracil), related compounds (e.g., 8-ethoxy caffeine), antibiotics (e.g., streptonigrin), alkylating agents (e.g., sulfur mustards, nitrogen mustards, epoxides, ethylenamines, sulfates, sulfonates, sulfones, lactones), azide, hydroxylamine, nitrous acid, or acridines. Further details of mutation breeding can be found in “Principals of Cultivar Development” Fehr, 1993 Macmillan Publishing Company the disclosure of which is incorporated herein by reference. See also Knudsen et al. (2021) bioRxiv 2021.05.20.444969; doi.org/10.1101/2021.05.20.444969.

The nucleic acid molecules, expression cassettes, vectors, and polynucleotide constructs of the present invention may be used for transformation of any plant species, including, but not limited to, monocots and dicots. Preferred plants of the present invention are wheat plants. Examples of other plant species of interest include, but are not limited to, peppers (Capsicum spp; e.g., Capsicum annuum, C. baccatum, C. chinense, C. frutescens, C. pubescens, and the like), tomatoes (Lycopersicon esculentum), tobacco (Nicotiana tabacum), eggplant (Solanum melongena), petunia (Petunia spp., e.g., Petunia x hybrida or Petunia hybrida), pea (Pisum sativum), bean (Phaseolus vulgaris), corn or maize (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), soybean (Glycine max), teff (Eragrostis tef), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa

(Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), palms, oats, barley, vegetables, ornamentals, and conifers.

In certain embodiments of the invention, the preferred plants are cereal plants. Such cereal plants of the present invention are grass plants (i.e. Poaceae family) cultivated for the edible components of their grain or kernels (i.e. seeds) including, for example, wheat, triticale, rye, barley, oats, maize, sorghum, millet, and rice. In certain other embodiments of the invention, the preferred plants are wheat, barley, and triticale plants. In yet other embodiments of the invention, the preferred plants are wheat plants.

In some embodiments of the present invention, a plant cell is transformed with a polynucleotide construct encoding an R protein of the present invention. The term “expression” as used herein refers to the biosynthesis of a gene product, including the transcription and/or translation of said gene product. The “expression” or “production” of a protein or polypeptide from a DNA molecule refers to the transcription and translation of the coding sequence to produce the protein or polypeptide, while the “expression” or “production” of a protein or polypeptide from an RNA molecule refers to the translation of the RNA coding sequence to produce the protein or polypeptide. Examples of polynucleotide constructs and nucleic acid molecules that encode R proteins are described elsewhere herein.

The use of the terms “DNA” or “RNA” herein is not intended to limit the present invention to polynucleotide molecules comprising DNA or RNA. Those of ordinary skill in the art will recognize that the methods and compositions of the invention encompass polynucleotide molecules comprised of deoxyribonucleotides (i.e., DNA), ribonucleotides (i.e., RNA) or combinations of ribonucleotides and deoxyribonucleotides. Such deoxyribonucleotides and ribonucleotides include both naturally occurring molecules and synthetic analogues including, but not limited to, nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs). The polynucleotide molecules of the invention also encompass all forms of polynucleotide molecules including, but not limited to, single-stranded forms, double-stranded forms, hairpins, stem-and-loop structures, and the like. Furthermore, it is understood by those of ordinary skill in the art that the nucleotide sequences disclosed herein also encompasses the complement of that exemplified nucleotide sequence.

The invention is drawn to compositions and methods for enhancing the resistance of a plant to plant disease, particularly to compositions and methods for enhancing the resistance of a plant to stem rust caused by Pgt. By “disease resistance” is intended that the plants avoid the disease symptoms that are the outcome of plant-pathogen interactions. That is, pathogens are prevented from causing plant diseases and the associated disease symptoms, or alternatively, the disease symptoms caused by the pathogen are minimized or lessened.

As used herein, a “race” refers to any of a group of laboratory isolates or fungal individuals existing in the field that share a similar virulence phenotype on a range of different resistance gene lines of wheat and are likely derived by clonal reproduction.

As used herein, an “isolate” of Pgt refers to a line of Pgt originally isolated as spores collected from an infected plant in the field or in a laboratory/glasshouse setting. Such an “isolate” is subsequently maintained in pure form by infection and re-isolation of spores from a susceptible plant and storage of said spores.

The present invention encompasses the nucleic acid molecules and polynucleotide constructs disclosed herein or in the accompanying sequence listing and/or drawings including, but not limited to: nucleic acid molecules and polynucleotide constructs comprising the nucleotide sequences set forth in SEQ ID NO: 1, 6, 7, 8, 9, 10, 11, 12, 13, 14, 16, and/or 17; and nucleic acid molecules and polynucleotide constructs encoding one or more proteins comprising an amino acid sequence selected from the group consisting of the amino acid sequences set forth in SEQ ID NOS: 2, 3, 4, 5, and 15. The present invention further encompasses plants, plant cells, host cells, and vectors comprising at least one of such nucleic acid molecules and/or polynucleotide constructs, as well as food products produced from such plants and plant parts. Additionally encompassed by the present invention are uses of plants comprising at least one of such polynucleotide constructs in the methods disclosed elsewhere herein such as, for example, methods for enhancing the resistance of a plant to stem rust caused by Pgt and methods of limiting stem rust in agricultural crop production.

The following examples are offered by way of illustration and not by way of limitation.

### EXAMPLES

### Example 1: Cloning and Characterisation of Stem Rust Resistance Gene Sr43

Sr43 is a stem rust resistance gene conferring resistance to Puccinia graminis f. sp. tritici (Pgt) isolate 04KEN156/04 (race TTKSK; also known as Ug99) and other races of Pgt (Niu et al. (2014) Theor. Appl. Genet. 127:969-980). We used the chemical mutagen ethyl methanesulphonate to mutagenize 2700 seeds of a wheat-Thinopyrum ponticum introgression line containing Sr43 (Niu et al. (2014) Theor. Appl. Genet. 127:969-980). The M₁ seeds were grown in a greenhouse, individual spikes (families) were bagged and the seeds of M₂ families were phenotyped with Pgt race TPMKC (isolate 74MN1409). The M₃ progeny from the susceptible M₂ plants of segregating families were phenotyped to confirm that the M₂ susceptible plants were true mutants. Eight independently derived mutants were selected for MutChromSeq (Sánchez-Martin et al. (2016) Genome Biology 17:221, doi.org/10.1186/s13059-016-1082-1). The flow-sorted DNA from the mutants were amplified by multiple displacement amplification and then sequenced. The flow-sorted DNA from the wild type was used to construct two PCR-free libraries and then sequenced. The sequence reads from the wild type were trimmed and then assembled with the CLC assembly program. The sequence reads from the mutants were trimmed and then mapped to the CLC assembly of the wild type. Using the ChromSeq Mutant Hunter pipeline (Sánchez-Martin et al. (2016) Genome Biology 17:221, doi.org/10.1186/s13059-016-1082-1), one contig was identified which had a different single nucleotide transition mutation in each of the eight mutants. The probability of this occurring by chance alone is approximately 1 in 550 million.

To determine the gene structure of Sr43, a higher order scaffold assembly of the wild type Sr43 flow sorted chromosome fraction was assembled using Meraculous. This generated a total assembly size of 1,263 Mb consisting of 168,523 scafffolds of longer than 1 kb, with an N50 of 13.9 kb. Then, total RNA from the wildtype Sr43 line was extracted. A full-length cDNA library was constructed and RNAseq was performed. The RNAseq mapping against the genomic scaffold sequences was performed. The RNAseq data was also assembled with the program Trinity to generate a cDNA assembly. The cDNAs corresponding to Sr43 were identified by BLAST. The RNA mapping indicated two or more alternative splice variants. The cDNA sequences at the 5′ end and 3′ end (polyA) were used to design primers for cloning different splice variants from the full-length cDNA library. Four different splice variants were identified by Sanger sequencing. All four transcripts were annotated and their respective coding (DNA) and translated (amino acid) sequences were determined. One of the four splice variants (the most abundant one detected in the RNA mapping) was found to cover all eight single nucleotide mutations. Note that the fourth splice variant is predicted based on partial Sanger sequencing results.

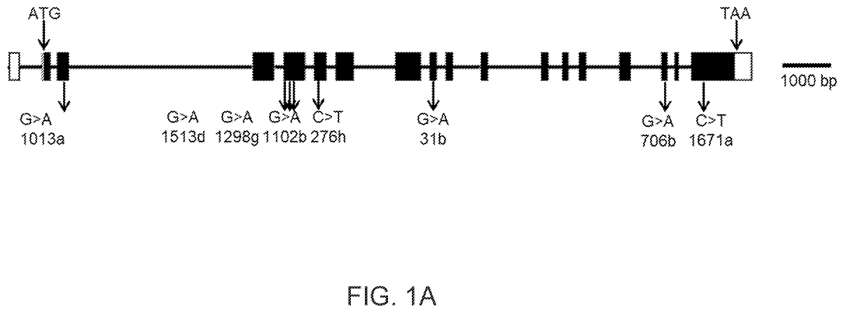

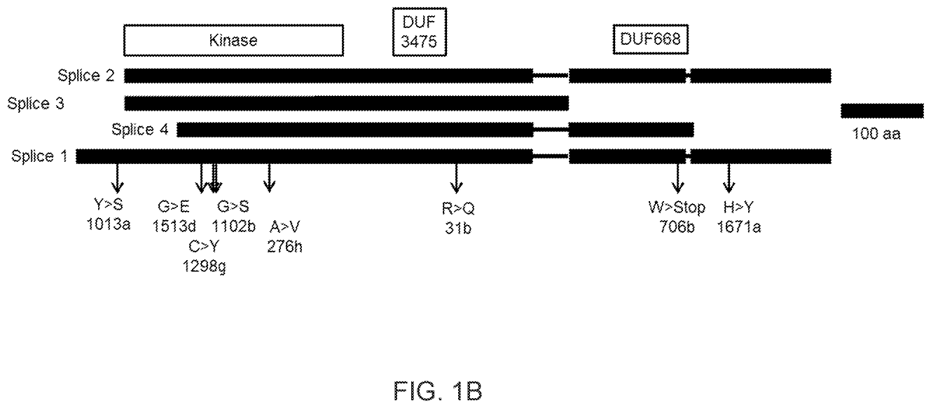

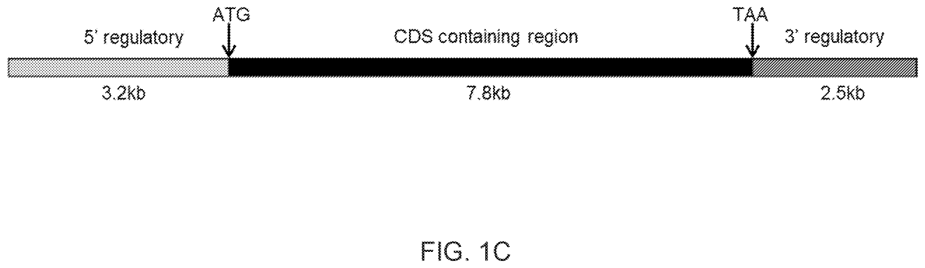

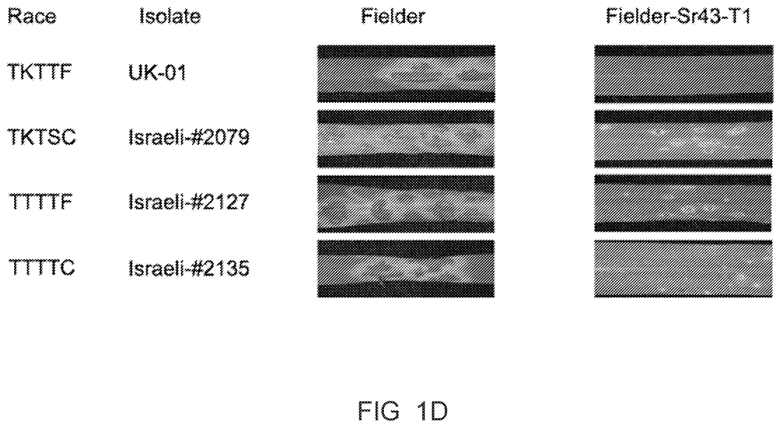

The most abundant splice variant, splice variant 1 (SV1), has 18 exons and results from splicing out 17 introns from the corresponding Sr43 primary transcript. SV1 has a coding DNA sequence (CDS) of 2,598 bp (SEQ ID NO: 6) and 5′ and 3′ UTRs of 128 bp and 221 bp (FIG. 1A). The CDS-containing genomic region of Sr43 spans 7,803 bp from the start to the stop codons (FIG. 1C). The predicted protein encoded by SV1 has 866 amino acids and contains domains with homology to a kinase (of the PKc-like superfamily) along with domains of unknown function (DUF) 3475 and 668 (FIG. 1B).

Based on the annotation, specific primer pairs were designed to amplify three overlapping parts of the genomic DNA sequence which cover the whole gene, including 3.2 kb of putative promoter sequence 5′ of the START codon and 2.5 kb of putative terminator region 3′ of the STOP codon of Sr43 based on splice variant 1. The three parts were cloned into the Topo vector and the respective plasmid DNAs were multiplied. The inserts in the plasmids were verified by Sanger sequencing. Then the plasmids for the three parts were digested with NotI (part 1), NotI and PvuI (part 2), and PvuI and PmeI (part 3), and the inserts were separated from the Topo vector by gel electrophoresis, cut out and purified. A three-way ligation was used to combine part 2 and part 3 in the pGGG vector (Hayta et al. (2019) Plant Methods 15:121) modified to introduce a PmeI and NotI site in the multi-cloning site. Following this, part 1 was dropped in at the NotI site, and a clone with the right orientation was selected to make the 13.5 kb construct of the Sr43 genomic sequence (FIG. 1B). Sr43 was then transformed into wheat cultivar Fielder.

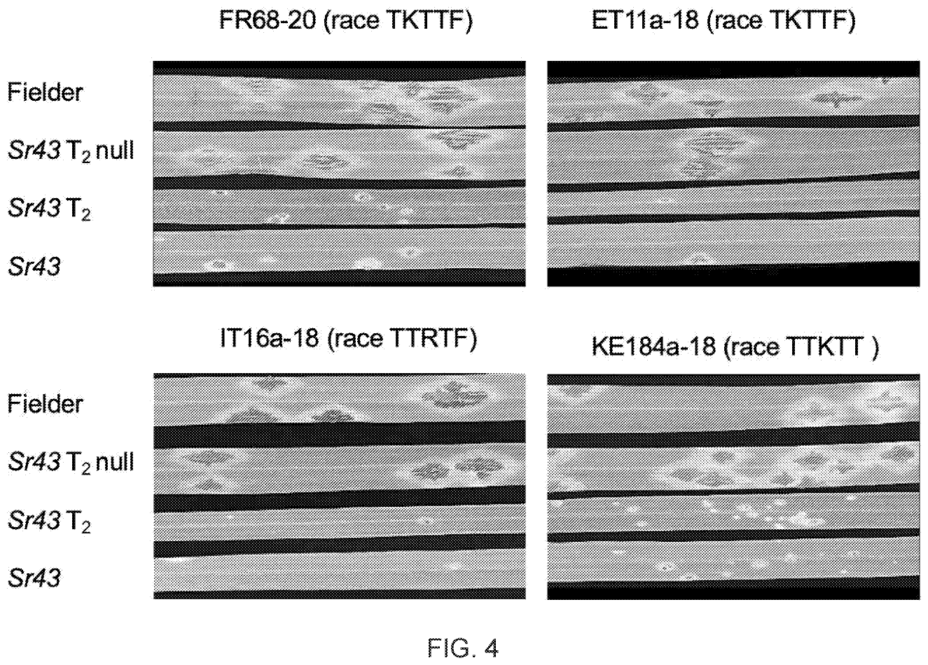

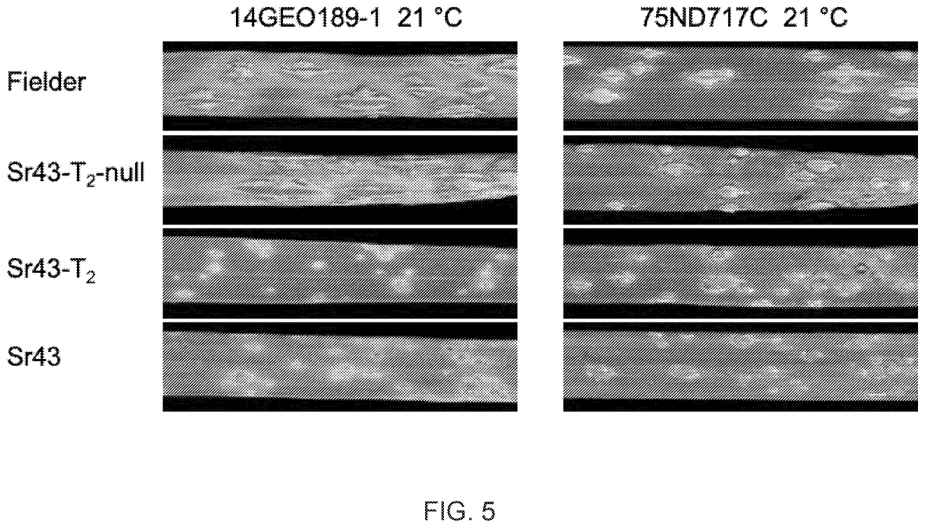

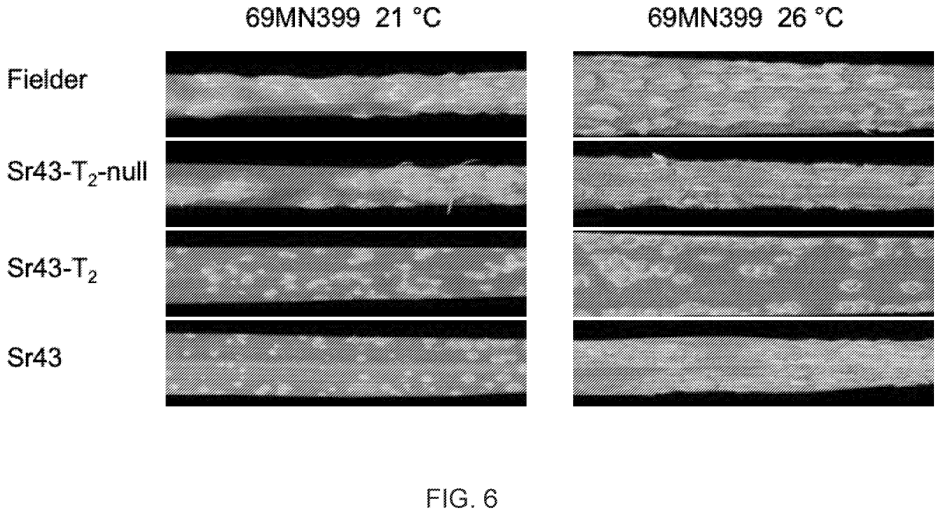

We obtained one primary (T₀ transgenic plant, which carried three copies of the transgene, based on qPCR of the hygromycin selectable marker. We tested homozygous T₁ and T₂ lines against a geographically and phenotypically diverse panel of 12 Pgt isolates from North America, the Middle East, Europe, and Africa (Table 1). In 11 cases, the Sr43 transgenic and wild-type introgression lines were resistant, whereas the cultivars Chinese Spring (the introgression parent) and Fielder were susceptible (FIGS. 1D, 4, 5). By contrast, the Pgt isolate 75ND717C was intermediately virulent on the Sr43 introgression and transgenic lines (FIG. 4). For Pgt isolate 69MN399, we compared the phenotype at 21° C. and 26° C. and noticed a marked reduction in Sr43-mediated resistance at the higher temperature (FIG. 6), in line with previous observations of Niu et al. ((2014) Theor. Appl. Genet. 127:969-980).

TABLE 1
Stem rust isolates used to test the
resistance of Sr43 transgenic lines.

| Entry | Isolate | Race | Origin | Year |
|---|---|---|---|---|
| 1 | 74MN1409 | TPMKC | USA | 1974 |
| 2 | 75ND717C | QTHJC | USA | 1975 |
| 3 | 69MN399 | QTHJC | USA | 1989 |
| 4 | ET11a-18 | TKTTF | Ethiopia | 2018 |
| 5 | KE184a/18 | TTKTT | Kenya | 2018 |
| 6 | IS#2079 | TKTSC | Israel | 2022 |
| 7 | IS#2127 | TTTTF | Israel | 2022 |
| 8 | IS#2135 | TTTTC | Israel | 2022 |
| 9 | FR68-20 | TKTTF | France | 2020 |
| 10 | IT16a-18 | TTRTF | Italy | 2018 |
| 11 | UK-01 | TKTTF | UK | 2018 |
| 12 | 14GEO189-1 | TTRTF | Georgia | 2014 |

### Example 2: Identification of Stem Rust Resistance Gene Sr62

Sr-1644-1Sh (Yu et al. (2017) Theor. Appl. Genet. 130:1207-1222), now designated as Sr62, is a stem rust resistance (Sr) gene conferring resistance to Puccinia graminis f. sp. tritici (Pgt) isolate 04KEN156/04 (race TTKSK in the Ug99 tribe) and other races of Pgt. Based on QTL mapping, two genes were identified in a recombinant inbred line population between Aegilops sharonensis accession 2189 (susceptible) crossed to Ae. sharonensis accession 1644 (resistant) (Yu et al. (2017) Theor. Appl. Genet. 130:1207-1222). We developed F_{3:4} families to genetically isolate Sr62 on the short arm of chromosome 1. Among them, family #803 segregated 3:1 for resistance and susceptibility conditioned by Sr62. We derived two large Sr62 segregating populations from #803 for use in high resolution mapping of Sr62. We also generated a high-quality chromosome-scale assembly of Ae. sharonensis accession 1644 (N50=12.3 Mb) based on the TRITEX pipeline (Monat et al. (2019) Genome Biology 20:284. doi: 10.1186/s13059-019-1899-5). The analysis of 9,276 products of meiosis (gametes) allowed Sr62 to be delimited to a 480 kb physical interval within the reference genome assembly. Based on RNA mapping with RNA-seq data obtained from leaves of 3-leaf stage seedlings of Ae. sharonensis accession 1644, we identified seven transcribed genes in the interval. The near-full-length cDNA sequences for the seven genes were determined based on the RNA mapping.

To further determine the candidate gene for Sr62, we mutagenized 3,025 seeds of an introgression line containing Sr62 derived from Ae. sharonensis accession 1644 in the hexaploid bread wheat (Triticum aestivum) cultivar Zahir background (Zahir-1644) (Millet et al. (2017) Plant Genome. doi: 10.3835/plantgenome2017.07.0061) in order to isolate mutants that lost resistance to Pgt race TTKSK and were therefore putative mutants in Sr62. The M₁ seeds were grown in the greenhouse and seeds harvested to generate M₂ families. The M₂ families were phenotyped after inoculation with Pgt race TTKSK and susceptible plants were identified and seed harvested. M₃ plants derived from susceptible M₂ plants were also tested to confirm that the M₂ susceptible plants were true mutants. Fourteen susceptible mutants derived from independent M₂ families were selected for further analysis.

We used the mutants for RNA-Seq to identify the gene Sr62. Total RNA was extracted from 3-leaf stage seedlings of the susceptible mutants and the wild type Zahir-1644 parent line. The raw RNAseq reads from the wildtype were trimmed and then assembled with the CLC program. The raw reads from the 14 mutants were trimmed and then mapped against: (i) the near-full-length cDNA sequences of the seven genes located in the genetically defined 480 kb physical interval in the Ae. sharonensis accession 1644 reference assembly, and (ii) the CLC cDNA assembly.

One cDNA contig in the Sr62 map interval was identified to have single nucleotide mutations in seven out of the 14 mutants (WTK-A, Sr62); Table 2). Another gene had mutations in six of the 14 mutants (NLR; Table 2), while WTK-B, TOE1-B1 and 50S had two, one and zero mutations, respectively. All the identified mutations were G to A or C to T transition mutations which are typical of EMS mutagenesis. The RNA-Seq coverage of the remaining two genes (Remorin-X2 and WAK) was too low to call mutations.

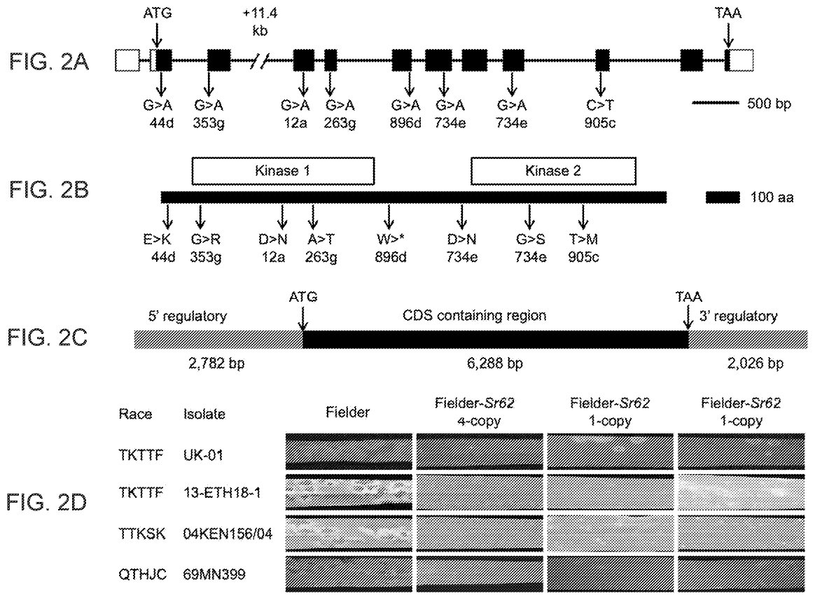

To study the predicted effect of the mutations on these genes, we prepared and sequenced a full-length cDNA library of the wildtype, and then we assembled the sequenced full-length library with the program CLC. Based on BLAST, we obtained the full transcripts for Sr62. Using the full-length transcript sequences, we determined the intron/exon structure, predicted the amino acid sequences and then established the effect of the mutations on their amino acid sequences. In Sr62 all the mutations were missense (six) or nonsense (one). In NLR, there were only two missense mutations while the remaining were synonymous (four) or outside of the open reading frame in the terminator region (two) (Table 2). Moreover, these two mutations in the NLR terminator region were not proximal to splice sites and did not appear to give rise to alternative transcripts based on the RNAseq analysis. The two mutations in WTK-B were both missense (Table 2). Based on this analysis, Sr62 was deemed to be the best candidate for Sr62. The probability of getting seven mutations in the same gene out of 14 independent mutants by chance alone is approximately 1 in 11,000 (based on the Sr62 coding sequence length and an average mutation density of one single-nucleotide variant per 31 kb). However, the fact that only 7 out of 14 mutants had an identifiable mutation in Sr62 suggests that there may be a second gene that is required for Sr62 function. Based on protein BLAST and BLASTX against the NCBI protein database (non-redundant protein sequence (nr)), the Sr62 gene is predicted to encode a tandem kinase (TK) (FIG. 2B) that has 52.7% amino acid sequence identity to the protein encoded by the powdery mildew resistance gene Pm24. Pm24 is a wheat resistance gene that is known to confer to wheat plants resistance to powdery mildew caused by Blumeria graminis f. sp. tritici (Lu et al. (2020) Nat. Commun. 11:680, doi.org/10.1038/s41467-020-14294-0).

TABLE 2
EMS-induced mutations identified in transcribed genes in the Sr62 map interval and the effect of these
mutations on the predicted amino acid sequence in 14 independently derived susceptible mutants.

| | | | | | | | |
|---|---|---|---|---|---|---|---|
| Genotype | Remorin | WAK | WTK-B | NLR | WTK-A (Sr62) | 50S | EAT |

| | | | | | | |
|---|---|---|---|---|---|---|
| 12a | Low | Low | | G > A, Lys > Lys | C > T, Asp > Asn | |
| | expression | expression | | | | |
| 44d | Low | Low | | | C > T, Glu > Lys | |
| | expression | expression | | | | |
| 263g | Low | Low | | | C > T, Ala > Thr | G > T |
| | expression | expression | | | | |
| 353g | Low | Low | | | G > A, Gly > Arg | |
| | expression | expression | | | | |
| 905c | Low | Low | | G > A, Pro > Pro | C > T, Thr > Met | |
| | expression | expression | | | | |
| 734e | Low | Low | | | G > A, Asp > Asn; | |
| | expression | expression | | | G > A, Gly > Ser | |
| 896d | Low | Low | | | G > A, Trp > stop | |
| | expression | expression | | | | |
| 119d | Low | Low | G > A, Gly > Arg | G > A, Asp > Asn | | |
| | expression | expression | | | | |
| 267d | Low | Low | | | | |
| | expression | expression | | | | |
| 1298e | Low | Low | C > T, Pro > Leu | C > T, Ala > Ala | | |
| | expression | expression | | | | |
| 743a | Low | Low | | | | |
| | expression | expression | | | | |
| 190d | Low | Low | | | | |
| | expression | expression | | | | |
| 200h | Low | Low | | C > T, Arg > stop | | |
| | expression | expression | | | | |
| 1101a | Low | Low | | | | |
| | expression | expression | | | | |
| Wildtype | Low | Low | | | | |
| | expression | expression | | | | |

For engineering a binary construct containing Sr62, specific primer pairs were designed to amplify two parts of the genomic DNA sequence which cover most of the native gene, including 2.8 kb of putative promoter sequence 5′ of the START codon and 2.0 kb of putative terminator region 3′ of the STOP codon, but which leave out 11.4 kb of the middle of the 12.4 kb intron (FIG. 2B). The two parts were cloned separately into the pTopo vector and the respective plasmid DNAs were multiplied. Then the plasmids for the two parts were digested with NotI and EcoRI (part 1), and EcoRI and PmeI (part 2), and the inserts were separated from the pTopo vector by gel electrophoresis, cut out and purified. A three-way ligation was used to combine part 1 and part 2 in the pGGG vector (Hayta et al. (2019) Plant Methods 15:121) modified to introduce a PmeI and NotI site in the multi-cloning site. A clone was selected which contained the 11.9 kb construct of the Sr62 recombined genomic sequence. The insert sequence was verified by Sanger sequencing and named pGGG-Sr62.

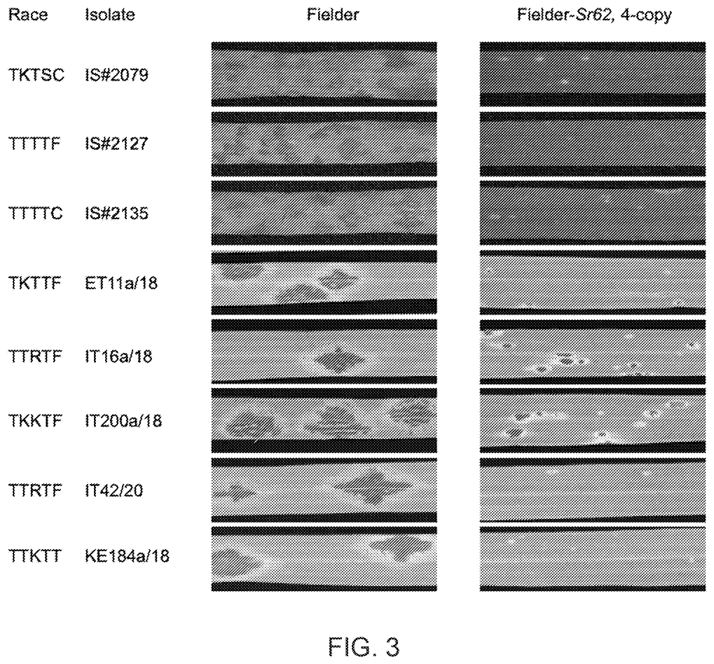

The pGGG-Sr62 binary vector was used for Agrobacterium-mediated transformation of wheat as described by Hayta et al. ((2019) Plant Methods 15:121). We obtained three independent primary transgenic lines (T₀), which, based on qRT-PCR of the selectable marker, were predicted to contain one copy of the transgene (two lines) or four copies of the transgene (one line). We advanced these hemizygous lines to the next generation to obtain homozygous lines. All three lines conferred resistance to stem rust races TTKSK (isolate Ug99, 04KEN156/04 from Kenya), TKTTF (isolate 13-ETH18-1 from Ethiopia), TKTTF (isolate UK-01 from the UK) and QTHJC (isolate 69MN399 from the US) (FIG. 2D). We also tested the line with four copies of the selectable marker against eight additional stem rust isolates/races from Israel (three isolates), Italy (three isolates), Kenya (one isolate) and Ethiopia (one isolate) and found high levels of resistance (FIG. 3).

The article “a” and “an” are used herein to refer to one or more than one (i.e., to at least one) of the grammatical object of the article. By way of example, “an element” means one or more element.

Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

1. An isolated nucleic acid molecule comprising a nucleotide sequence selected from the group consisting of:
(a) the nucleotide sequence set forth in SEQ ID NO: 1, 6, 7, 8, 9, 10, 11, 12, 13, 14, 16, or 17;
(b) a nucleotide sequence encoding the amino acid sequence set forth in SEQ ID NO: 2, 3, 4, 5, or 15, and optionally, wherein the nucleotide sequence is not naturally occurring;
(c) a nucleotide sequence having at least 85% sequence identity to at least one of the nucleotide sequences set forth in (a), wherein the nucleic acid molecule is capable of conferring resistance to stem rust to a wheat plant comprising the nucleic acid molecule and optionally, wherein the nucleotide sequence is not naturally occurring; and
(d) a nucleic acid molecule comprising a nucleotide sequence encoding an amino acid sequence having at least 85% sequence identity to at least one amino acid sequence set forth in (b), wherein the nucleic acid molecule is capable of conferring resistance to stem rust to a wheat plant comprising the nucleic acid molecule and optionally, wherein the nucleotide sequence is not naturally occurring.

2. An expression cassette comprising the nucleic acid molecule claim 1 and an operably linked heterologous promoter or a vector comprising the nucleic acid molecule or the expression cassette.

3-5. (canceled)

6. A host cell transformed with the nucleic acid molecule of claim 1.

7-10. (canceled)

10. A plant or seed transformed with the nucleic acid molecule of claim 1.

11-12. (canceled)

13. A transgenic plant or seed comprising stably incorporated in its genome a polynucleotide construct comprising a nucleotide sequence selected from the group consisting of:
(a) the nucleotide sequence set forth in SEQ ID NO: 1, 6, 7, 8, 9, 10, 11, 12, 13, 14, 16, or 17;
(b) a nucleotide sequence encoding the amino acid sequence set forth in SEQ ID NO: 2, 3, 4, 5, or 15, and optionally, wherein the nucleotide sequence is not naturally occurring;
(c) a nucleotide sequence having at least 85% sequence identity to at least one of the nucleotide sequences set forth in (a), wherein the nucleic acid molecule is capable of conferring resistance to stem rust to a wheat plant comprising the nucleic acid molecule and optionally, wherein the nucleotide sequence is not naturally occurring; and
(d) a nucleic acid molecule comprising a nucleotide sequence encoding an amino acid sequence having at least 85% sequence identity to at least one amino acid sequence set forth in (b), wherein the nucleic acid molecule is capable of conferring resistance to stem rust to a wheat plant comprising the nucleic acid molecule and optionally, wherein the nucleotide sequence is not naturally occurring.

14. The transgenic plant or seed of claim 13, wherein the polynucleotide construct further comprises a promoter operably linked for the expression of the nucleotide sequence in a plant.

15. (canceled)

16. The transgenic plant or seed of claim 13, wherein the transgenic plant is a wheat plant and the transgenic seed is a wheat seed.

17. The transgenic plant or seed of claim 16, wherein the transgenic plant or seed comprises enhanced resistance to wheat stem rust caused by Puccinia graminis f. sp. tritici, relative to a control wheat plant.

18-19. (canceled)

20. A method for enhancing the resistance of a wheat plant to wheat stem rust, the method comprising introducing a polynucleotide construct into at least one wheat plant cell, the polynucleotide construct comprising a nucleotide sequence selected from the group consisting of:
(a) the nucleotide sequence set forth in SEQ ID NO: 1, 6, 7, 8, 9, 10, 11, 12, 13, 14, 16, or 17;
(b) a nucleotide sequence encoding the amino acid sequence set forth in SEQ ID NO: 2, 3, 4, 5, or 15;
(c) a nucleotide sequence having at least 85% sequence identity to at least one of the nucleotide sequences set forth in (a), wherein the nucleic acid molecule is capable of conferring resistance to stem rust to a wheat plant comprising the nucleic acid molecule; and
(d) a nucleic acid molecule comprising a nucleotide sequence encoding an amino acid sequence having at least 85% sequence identity to at least one amino acid sequence set forth in (b), wherein the nucleic acid molecule is capable of conferring resistance to stem rust to a wheat plant comprising the nucleic acid molecule.

21. The method of claim 20, wherein the polynucleotide construct is stably incorporated into the genome of the plant cell.

22. The method of claim 21, wherein the wheat plant cell is regenerated into a wheat plant comprising in its genome the polynucleotide construct.

23. The method of claim 20, wherein the polynucleotide construct further comprises a promoter operably linked for the expression of the nucleotide sequence in a plant.

24. The method of claim 23, wherein the promoter is selected from the group consisting of pathogen-inducible, constitutive, tissue-preferred, wound-inducible, and chemical-regulated promoters.

25. The method of claim 20, wherein the wheat plant comprising the polynucleotide construct comprises enhanced resistance to wheat stem rust caused by Puccinia graminis f. sp. tritici, relative to a control wheat plant.

26-29. (canceled)

30. A method of limiting wheat stem rust in agricultural crop production, the method comprising planting a wheat seed according to claim 16 and growing a wheat plant under conditions favorable for the growth and development of the wheat plant.

31. The method of claim 30, further comprising harvesting at least one seed from the wheat plant.

32. (canceled)

33. A human or animal food product produced using the wheat plant or seed of claim 16.

34. A polypeptide comprising an amino acid sequence encoded by the nucleic acid molecule of claim 1.

35. A method for identifying a wheat plant that displays newly conferred or enhanced resistance to wheat stem rust, the method comprising detecting in the wheat plant the presence of an R gene comprising a nucleotide sequence selected from the group consisting of the nucleotide sequences set forth in (a)-(d) of claim 1.

36. The method of claim 35, wherein the presence of the R gene is detected by detecting at least one marker within the R gene.

37-39. (canceled)


SEQUENCE LISTING (17 sequences)

SEQ ID NO: 1 - DNA, 13533 bp, Thinopyrum ponticum
  source 1..13533: mol_type = genomic DNA
tgtaggcacacaagtaggggaggtgtagttcaactcatgatctatcatatccctactagttcataaattgatcatatctttcacatccaacatttatgatgtcacatgtgcttcaaagatgaatatttgttttgggaccaatgtttatatcttcgcggtgccaagccaaacatactcaaacatggtggctacggccaccgtcctcctcttgaggaggttggatcttaatttgcattttatttgggtattctacaagtcaactataggctaaatatgatcatacactcatcatttggtgtttttttcgttcgtgcaaatatgagtgcataagatctctatacacccttttagtgtgattctatcaaatgccttcttatctaaaagacatgttttcacctcttgcaagtgttcctcttggcttaaactcaagtagaattggatttcattgcgacaatgtccttttgtgtgagggctctacagcttggaacttgtgaaggtcagacaagtctaaccacctaagtcccttttcctatactggtctcaaatgcttgtggacaccccgtgcacatgagcttgtaccatgcgcatggggccccatgcacatggggttgaggttccaggcggggtatgagtaggcgcgcggggtggttagggttccccactccacatccaacccccacgcgcccagacgactcactccgctgccaccgttgcctccttcatctccgacgagatcacgtacccacaggcctctccctcggcatcctcttgctggaatggggagctcctccggtacgtgatcccgcgcctcttattcattcgatgaaactcctcctttttcctccatatattttagggtttcgtccactgctcgggtagattcacataatctaggactaaattggaatagaaactgctaggaaccatctggatgggtatagccgcattcactcccgtggatttatccttggagtggccatggctggagcaccccatgtgcacgggcttgtaccatgcacatggggctcacctcccgtgcacacatggtatccagaggagttccacatgtttcttttttagttttgttcttctgcacataaccaaacacttgctcttatcttctctgtctatctatctctctcagatatgtccgctagcaccgtggaggcttctcatagcaagaagaccaacccctccgagagcgcctccaacttcaccatctgttgtcgtgagccagtggattcttctgcagtccatcagtcagacggtgtacgcagggcaaccaacaagaaggtcgatgcagcttctcatggtgttgagattcagcctggatctgaggacgtggatagctaggctaccgactatgataacccactagcaggatggcgctttgatgatgaccaggatgatctcacgagtggccctctgcgtgtctaggtcacgttagcctctcctgttccctttttggcgtcttgatgccaaagggggagaagtgttctatataggagcatttgtatttgggagatttcaagccttgttcttcattgttggcttttatgtggcttgaaatacttttattgatgtgagtcacttttttgtgatgatgctatctttatgacttggtatgctatgcttattgattccatgtgatcatatcatgtcatctattgctcatagtctttgatatattgctctcacaccacacctcttgactgtgatctctagcacaagtggtcgccggttccagcacgagtggtcaccggttccagcatccaccgcgtccggttctagcaatgttgcaacgccgtcgaagcacgaccgcacgccgggtccagcacgaccggttccagcatcaccgtgtgcacgttccattaatgctacaactccgtcgaaccacggccgagcgccggttcccgcacgaatgccgtgcccgttctgccggcggccatccgtcgtaacatctccgtggcctcgcgtcgcagcgccggtgggctctagcatcgcagcaccggtggctgcccgtcgcagcggtgccggtggacgctcgtcacagctccaccccttgtagcagcgcctgcgacaagccggcaacctgaaaacgtgcggcgtctggggattttgcagcagcgcgcagcccctgtagcgcggcggtcaggctcgagatagcgggggattcaggcttgcgcgcggcggtggtcaaggactcggcggcagtgaggattcagcatgcacggcatgctcccagctgcggtgcgcaagatgagaggaaggaagcgacgagatagaggagatgcaggaggaagaaacgagtggttcgtctggggataaggttgttgcgaaagcggtggaagcagggcccacagaaggacacgcggccgcgcaaaagtggaggaccggctacgcgtgggaaaggcaggaaggatgctgagcgcacgatctcattgatcgagcggtgcaagcgcgaccggcgagagtcaaggccggtcggctgatacagtacccgcgagaggcgaaatgaaaagcgtcgagtcccctcgttccgccggcgaccagaccgagggttccctccctctccgtccgctgctccagcagcgcgaaccccggatctgtgtctccggtgtgtacacagtagggtaggtgctcggatggtggcgcggtggggccaaacgcggcgtaaaaaaatcagggggtatgtacatatatcttactggatgcagaacaaaaacagatgcatacaaagacgagtggacgaaaaatccaaggtcatggtagctttcactttcaggcagacgagttagtgggcgacatccaccggattcagataaccacaaacactatagaaatagaataaagtccccaaaagtccaaacaatgaattgatcagctcatcatcgtcgcaaggagctgtagcccaagaggccaagaccatcacgagctggcggttcattcccgttccaggagcgttgaccagctccacatcgttcctcacagcagcctttttcaaggttagtactagtgctttgctctctgcctccctcccagacgtctcctactgcttgctttaattcgtaacctgtggttaaattcatgcttctttgctcttcaagatcggagctcgtttcaggcccagtctgcgaacccacccaatttcccccttctgtggaactcaaccgtttgatacacatggaaccatagcggagatccatatacacgtttaattttgtattgtattgtgtttccaggcatggataatatatttcgcaaattcggatggaaagctggtagacggcaacaaggagaaaggcagtttgattcgccatgagtctcgattccttatacaggcataaaaatttctctgatgtgacgagtgaaattgctatatttatttttgcctaccaggagagggaatagcacgggcagggcgtcgccaaggcctctttcgtaccatttattaggagaaattacaaatggtttctcggagaaccgaaaacttggtactggtgcatatggaagtgtttacaaggtaagatttcagccacaatctcatgaagttagacatggggtggtacttgtggatgcacaccacacattatttggtcaacaacttgctattcaccagccactacaccattattcagcggttaacgagagtaccatgcatgtacaaccattaaatgcatacagaagctaactcgtgtatataaatacaggcacattcaagttcacaggccacacatatatcttttgatctttttttttttttttttgcgagaaaatttccaatctattaatattcaatcatggcagtacaacgaatatcagaaataatggaaattacatccagatccgtagaccacctagcgacgactaccagcactgaaacgagccgaaggcgcgccgccgtcatcgcccctccatcgccggagtcgggcacaacttgttgtagtagacagtcgggaagtcgtcgtgctaaggccccgtaggaccagcgcaccagtactacaaccgtcgcagatgaagaataacgtagatcagaaagatccaatccgaaaacacacgaacgtagacgaacaacgacgagatccgagcaaatccaccaaagatagatccgccggagacaaaactccacacgcccaccaatgatgctaggggcaccgccggaacgggggctaggcggggagacctttattccatcttcagggagccgccgccgtctcgtcttcctgatcaggacacaaaccctagcaaaactgaaagaaacaactaaaaacggagccctcccgccggccattgccgagatccaccacgcccccatggcccttttgatctactgatcttgttaatgactagcatgcatacccccgcaaaaaaaaaaaatgactagcttgcatacttggtcttgcgccgctgtgcaattttacgccatataacaacaatacaccctcggtcccataacatggtgcacatggttttcgcgaaagtcaatcttcacaaaatttgaccaagttaattgagaaaattgtctatgtctacactaccaaatatataccatatgaaagtatatttcatagtgaatctaatgatattgatttggtattctagacgttgataaattttcctataaacttagtcaatgtttacacagtttgactttgaaaataatctaatacacactattttttgggggacggagggattataagggcatttctaaccaatcccttataacatgttagcctctaaccgggacctaaaaaatctattcggccgccgcaaatctcactatttttactccaccgctcggcggccgagtaaaaatctctccctcgcctctgtcgcctcttctcccctcgccctgccgccggcgcgtcccccgcccttccctgccgccccctctcctcccaatggccggacacggtggccggaggtcccctccgccacgccggcgggtcgtcgtccgccggtggatcctcgatgcgcccgccctcacccggttcgccgatttcccggagctccctccgctgcggccgtcgcagaggacctacctcggcgtccgacaacgctcgtgggggacatgggtggtggagatcaccgacctggagacccaccaatggaagtggattgggtcgttccacacggcagatctcgcggccatggaatacgactggtggtaagtccggtaccacggctccgtcgcccggctgaactttccattcgggacggcgccggtccaccttcttccgctagagtcgtgtgtgctgagcgcggcgatagcgtaggaggaccgagagccgagggagtgcctcgaggtggaggccgccgctgaggcgtacatggatgacctccgccgctagcacccggagctcgtggaggcggagcggctgatattcgccgacaatggcggggaggtcatcgtcatctccgacgatgaggtggaaggaggcgaggagggcagcaagaatactgcgctattttttaggggatcaattagaaatgccctaacaatagagaatacgggtcaacatatattctataatgagtattaactcattctgctctgtacttgaaacttataccgctacaaacaggaaactaaaccattcttgtgaaatattgaatagggggagcataaagacggggaaaagattgctgtgaagatgcttcatgatactctaggacttgacaatgagcaatttgaaaaggagtattttaaccttgcagatctacagcacaaaaatattgtccgattagttggctattgccatgaaactcggcgagaatgtgtgccttacaacggaagaatggttttcgctgacataacaaagagagcgctttgctttgagtatatgcgtaatggaggtcttgacaaatgtctttcaggtataatctatgggtaatctacttgtattaggaggtagaaatgcaaggaatagttattaatatacgtttcgcggatattttctatgtttatgcagatgaaactagtggacatgattggtgcacacgctattcaatcattaaggggatttgcgagggtctgaaatacctgcacgaggaattggaatctcctatgtatcatttggacttaaaaccagccaatatattactcgatgagaaaatggtgccgaaaatcgcagatttcggcttgtcaaggttcttcagaggagagcaatcgcaaatcacaaaaagtgctataggaacacagtaagcttacactatcatgtgaactgaaattttctttagcaggaaattctgtttgcactaaatatacggcctttttttaaacatatatcctgtatcatgcagtggatacgtaccgcccgaatacatagatgcatctgtactctcaattaagtttgacatattcagcttgggtgttgtaatcataaagataatgactggaccaacaggctacttcagaagtgctgaaatgtctgcaaaacaattcatagaacttgtaagcaaaattgttgcctttacagatatcaatagtaaatatttccgaattaaactgccatctttactgatatactatggttttttatgttccgtactggtaggtactggcaaactggaggaagaggctacctgcaacatcagtgtacctgctggagtcatactcggaacaagtaaagaggtgcaccgcaatagctgtaagttgtgtggaggctgatcgatccaaaaggccgtctataggggaaattgtgaataagctgaatgagacagagacaatgattcaaaatgtggcaaggaaacatacacgatcgtcggtggaccaggtgtgatcactaatggcacaagttatttacttgtgttttctattttcactataaaataaaataaataaagtgaaattagcacgcatgcatgtatgtatacagagagtagtgctaatataccccagtactgaataaaccatttaatgtttatagcgatgcacatagcaatatttcataattttgccaccacattagtaagtccagcataaccagttcgaaacgaacatgtcttcctatgttagatgaacatgtcttcctatgttagattgttagtttctttgtataagctagcacatgttattgtttcttataagggacaagacggcctcctttcttccctttgcagtgtgctataacttaccaactttcaattcattgttcaaaacatagaaatcagagattattacctgagaagacagcagtatccttttccaattagctctaacatactattttatactaggtatcagaaccagttacatttataggaggagctgttggggtgttagatacccttggcggtgttagcttaaataataatcatggcagtggtttcttgggcggaggaacaaatcgaggaagtaaagtcagtattttggcatttgaagtcgcaaatacaatagctaaagcttccgatttgtggagatcatgctctgacgagagcataaaagaactgaaggaagaaattttgcattcagatggtgtgcgaatattaatttcctcgaattccagtgagctcctgcatattgctgctatcggcaagaggtatggttagtttcaaagtaagttttttttattcatcttactgtaaacgttgttgatgtggatggaacccaacagggaagaactcgccatcctttcaagagaagtaattcgatttggtgacctgtttatagaccccatatggcataacttgagacgctattttgaaaagtatgcttctgagaattatgcatttcatttcgacatgtcattatttaccgtcttatcagcacctcttgttctgaacactatgggttactctggtgtgcaggtcaacgaaagattccatgccccaggatcattcaaaagagcatatcggaactactgtccagcagttgattagtttggctcaaaacacttctgtatgttctacttaatttccacattcctatatttcagaaggtgcttgtacttgataataatactaacgtttatggagtcaagaagtggcatcatcattacaactgtacttcttgaaattattgaacggctaaacatctttaatgaaatcaagaatctttcagcaggttaataaccaagaatcaggtctcttgtatatcagccagctagaactacagtctgagggtggatcaggctatttttttgcattcatactctggcttgaagcttatgctgatgagctagttctataactgcagaagctttaccatgaattgcacgctctggatagattggcgcaggaatttcaaaggaaatttcaggaagaggagtctgtaccagcaactagacgaggtacaaccagactgatgacattgttcttaagcaacttcagtagatgctaaaactgaatgcatattgcttttccagtatctcacccggaaataacgactgtactacataaattaagactttagtgaagagatcttaatacagtttttttactacgagcactgtagttttccatcaaaaggggaaaatgatgcttttgttggttgtgatgcatttttttactctgaacgttctctgctgatattttatcttacctattgccaatccaaagctccaagtgaacaaaattttgacagctgctgtgattattgataaatagtggtaaaccaagcatcacagctaaggttcttacatggagaaccagggcctagaggaaaatttcacatttttagataatacaagaaatgccaatttatcttcctggcaatgccattggaggcattctgataaatatttaaatatctttgtcacagtatacttttatacagcctttttttatagatgatatgatattaatttaagacaccatgtactgcaaatttgctactattagagatatttattgtattcaatttttgctctttgcagagagtgttatgattttgcacagtgaactaaagcgccaatggaagcttgtgaaaaatttgaagaagaaatccttgtggtccagacctttggaggatgtaattatcttcatccacatttgcttcattacttcggccttctgtaattctgtctgttgggttatgcctggcagcgttttgttgagatagtttttgatgaatgcgctactggcatatttgctttcttttgatggatgctgacattgctttgtgttatcctctatccagattgtggaaaagcttgttgatatcgtcagttttctagataaacaaatccgggatgcatttggtgaagccggtaattgattattgattggctgctttattgttcgcagaaaatgtatccgagtacctgacaatatcctgacacctctctgaataaacagttcctgtaggtactgacttcatcgagcaagttcagaacaaaaggctaggttcctgtggtctggcactacattacgctaacattatcaatcaaattgaaaatatagtaagttaatttgacgacccatgtcatgtttattgtgtttgcttaatgtagccaatttccatgtgacctcttatttatctatgaaactatttctgttatgtcgatgaaccgtgcatttatttatttccctttcctttcggtgaacagaagtgttcaatagtgatctgtaatgaatattttatggtcacctgcataaatatcagtctatctcctagctgcaaaataccaatccgaaagtaatttttgaaataaagaagttacaacgtcatagtgcatatattgtatcaccgaagtctacatgcttcagtctacgtcgttgcttaagaaataagaaaaaggcatagagttctcaatcttttgtgtacatatgcaggtttctcggccactctctcttcctcctagtgctagggacaacttgtaccatggactaccagtaacagtgaagtcagctctgcggtcaaggttgcaatcagttgatactgaagaggaggtttgtcttacacaactcggcagaatatttatttaattatgcacatggaatatatagtagaaacttataactggtggttgagcttagccaccagaatactgttttatgtctgtccagacatgattcagttctaagcactgaaagtttagttgtccatgtgggcagtgtaaccacatgatatccaaaggagcaaatacatgggatacatattgtagaatgtgataggcattgtgcatatattttcttgacaagagccaggacttgattttgttgctattattggtacatcaaagcgtatgactgaattgcgatcctttttttattctctgcagcatactgtatctcaaatcaaagctgaaatgcagaaaacccttcgctggattctgccaatagcagaaaatacaaggaggtacttcaaatcattctgatttctgagcatagttgtttttctccttgtttacaaaattcactgtttccatacttgtacaagagcacaccaagtatttggatgggtcggcgaatgggcacactttgggttagtctcctcaaatatcatgatttcctcatcttatcccctttgaatttctatcgaatatatcgtatacacttgtttgcaacataacctttttttttttttttgcgaatttgtttgcaacataacctgacgtagagattttacaggagtgacatggatgagaagtcaggttcccggcacagcgtgatcggggtccagacgcttcatcatgccgacaaggcgaagacggaggagcacatcctcgagcttgtggtgttgctccatcatctcgtggtccaggtgaaaagccgaggctacgagcacaacaattccacaaagcaggaccggtctcggtcctgcaaggtatcatcggaaccgcaagccgatgccaagcactacacatctccgacgaacaactgtgagatctgcccgagcccgctgtcggactccgagcgccggacgctggaccacctgagcttcaagagctgcgagcctcagcctggcagggtggacaaggcgcataggagcggggactcgtgccggagccatggcagctcgccggcgagggagttcagtcgtagcttgacctcgggccgtgagctggtgaaggatctggatgtaatagatggtctagcagactgactatcgtttagttaagagtgaagacatttggttgcacagcagagtaggtattctttggcctttgccgccgccttcttctatctcgtcgatcagatcgtatatatcttgtatgtgtgttttcgaccgttacatatgtagattatataaatgacagtgttacagctgggttgtgatttgaatggaaatatagcaagatcacaagtgatgctccattgcctccattgtgtcactctgtatactgcgagcatccacagtgtggtattttgctgcctctatccttgcctctaagctatttggagctgcctctatacattgcatggatgagcctctaagcaaaaatgcttggcgtcgcctctaaggtaagaggcagcctctaagccttttcttggactaaaataatggcatggccctatgggtgtgtacacataaaagctggctggtgaagttttgttgataaaataatattactttctactgtggtgggatccatgttagaggctgtatcaaagattgaacattgcaggttaggacgcttagcttagaggcagcatggtgggacccagcatagaggcagcagcgcctccaaacactgctgatgctttatgatacctgaattttcagtgtgtcggcagcttgcctttactagagtttattgtgactttctgaacacttgatcagtagaacctcccaataaaactgtttggatctttctcatcgaaggccaaataaaacctaggactccgcttgaaatggtttgtagaactagcgcggccaccggtactcaagtcaattttgaactagaaattagagataattgttccagagggcctgcatgcttgtgaagtcctcctctactcccaatctcccacgcatacgcatgctaccctttttcttcttcttaaaaaaagtgtgttcgcaaaataccatcgccatgattcggccggggtggtccagagcttatctactgctaactgagtactactactactgatagtgtcttctcttctcttcaccaggtcatccatcttatcaggatatcacatatcattatatcaacaataatgcaagctatctactccctccgttcctaaatatacgtctttctagagattccactacggactacatacggagcaaaatgagtgaatctacactctaaagtacgtctatatacatccgtatgtagtctggtttgaaatctttaaaaagacttatatttaagaacggcgggagtaaaaaataataataatgcaagcaagcacttttcccatacatatccgtaccctattcatttcacggcgacgtccacgtccatatgtccgccgctcccctcatccccagctctgccacccacgtgtacgcggcgccgagagagatgagaccccctgccgtacgtcgccgtcgacgtcgccatggcgcgcgtggcacggtcggcgtcggcgtctccgacgacccacccacccgccgaattgataacgccgtccatcacggctcggtctgccgccagctgcatgcatggccgactggcttcttgtcggcgctgcccgcctagacatgccatgtgcaagcctgagataggcagcgcataacacgtacgaccacaggagggcatctctgaacacgtaccatacgagtagcttcagcttcggagatactagcatgcatcttgaaccatctagaacaggaagaaaaacaggctaattaattcttggtggtgattggtgacaagacaaacgcggtgtttgtgtagaaaaatcagaggtactgtgtccatctggccttgacggagaacgtgatcagatacgaagaaacatgagtgcagaaaaatcagaggtcacggcagatcattgcgtgcagatgcatacgtagctatcactttcaggaagacagattactgggcgacgtgcaccggattcagataaccacaaacactatagaataacaaagtccccaaaagtccgaacgacgaaagatatgtgaccacacaatcgattagcccatcaccgttgcgaggagctatggcttaccttggagaacaaaggaaaaatctatgatctccatttggggcgtccaggaaaaatgtcatgaattcttgtaaaacaagtcaacgatatgaaaatagagattcgaaattcgaacgttccagataaacacaaacgctagaataaacaactagtccacaccgtgaaagaatatcatcaccatctgtcagcatcgcgagacctagctcggcaagaacaataacggaaagcctatcgttcttgttgggaatccaaacacaaagtctcatgtactattttttctaaaacaagttggccatattgaaccatagggtgacacctatgtatatgatggcaccgagatttcaaaattcaaattcgacatgtcagcacgccaagaaaacaaacagtactatccacagctccatctgccatcttgaccagaagctttttattttccgaagcacgaggaagactgagtgg

SEQ ID NO: 2 - protein, 866 aa, Thinopyrum ponticum
MDNIFRKFGWKAGRRQQGERRGNSTGRASPRPLSYHLLGEITNGFSENRKLGTGAYGSVYKGEHKDGEKIAVKMLHDTLGLDNEQFEKEYFNLADLQHKNIVRLVGYCHETRRECVPYNGRMVFADITKRALCFEYMRNGGLDKCLSDETSGHDWCTRYSIIKGICEGLKYLHEELESPMYHLDLKPANILLDEKMVPKIADFGLSRFFRGEQSQITKSAIGTHGYVPPEYIDASVLSIKFDIFSLGVVIIKIMTGPTGYFRSAEMSAKQFIELVLANWRKRLPATSVYLLESYSEQVKRCTAIAVSCVEADRSKRPSIGEIVNKLNETETMIQNVARKHTRSSVDQVSEPVTFIGGAVGVLDTLGGVSLNNNHGSGFLGGGTNRGSKVSILAFEVANTIAKASDLWRSCSDESIKELKEEILHSDGVRILISSNSSELLHIAAIGKREELAILSREVIRFGDLFIDPIWHNLRRYFEKSTKDSMPQDHSKEHIGTTVQQLISLAQNTSLYHELHALDRLAQEFQRKFQEEESVPATRRESVMILHSELKRQWKLVKNLKKKSLWSRPLEDIVEKLVDIVSFLDKQIRDAFGEAVPVGTDFIEQVQNKRLGSCGLALHYANIINQIENIVSRPLSLPPSARDNLYHGLPVTVKSALRSRLQSVDTEEEHTVSQIKAEMQKTLRWILPIAENTRRAHQVFGWVGEWAHFGSDMDEKSGSRHSVIGVQTLHHADKAKTEEHILELVVLLHHLVVQVKSRGYEHNNSTKQDRSRSCKVSSEPQADAKHYTSPTNNCEICPSPLSDSERRTLDHLSFKSCEPQPGRVDKAHRSGDSCRSHGSSPAREFSRSLTSGRELVKDLDVIDGLAD

SEQ ID NO: 3 - protein, 793 aa, Thinopyrum ponticum
MLHDTLGLDNEQFEKEYFNLADLQHKNIVRLVGYCHETRRECVPYNGRMVFADITKRALCFEYMRNGGLDKCLSDETSGHDWCTRYSIIKGICEGLKYLHEELESPMYHLDLKPANILLDEKMVPKIADFGLSRFFRGEQSQITKSAIGTHGYVPPEYIDASVLSIKFDIFSLGVVIIKIMTGPTGYFRSAEMSAKQFIELVLANWRKRLPATSVYLLESYSEQVKRCTAIAVSCVEADRSKRPSIGEIVNKLNETETMIQNVARKHTRSSVDQVSEPVTFIGGAVGVLDTLGGVSLNNNHGSGFLGGGTNRGSKVSILAFEVANTIAKASDLWRSCSDESIKELKEEILHSDGVRILISSNSSELLHIAAIGKREELAILSREVIRFGDLFIDPIWHNLRRYFEKSTKDSMPQDHSKEHIGTTVQQLISLAQNTSLYHELHALDRLAQEFQRKFQEEESVPATRRESVMILHSELKRQWKLVKNLKKKSLWSRPLEDIVEKLVDIVSFLDKQIRDAFGEAVPVGTDFIEQVQNKRLGSCGLALHYANIINQIENIVSRPLSLPPSARDNLYHGLPVTVKSALRSRLQSVDTEEEHTVSQIKAEMQKTLRWILPIAENTRRAHQVFGWVGEWAHFGSDMDEKSGSRHSVIGVQTLHHADKAKTEEHILELVVLLHHLVVQVKSRGYEHNNSTKQDRSRSCKVSSEPQADAKHYTSPTNNCEICPSPLSDSERRTLDHLSFKSCEPQPGRVDKAHRSGDSCRSHGSSPAREFSRSLTSGRELVKDLDVIDGLAD

SEQ ID NO: 4 - protein, 478 aa, Thinopyrum ponticum
MLHDTLGLDNEQFEKEYFNLADLQHKNIVRLVGYCHETRRECVPYNGRMVFADITKRALCFEYMRNGGLDKCLSDETSGHDWCTRYSIIKGICEGLKYLHEELESPMYHLDLKPANILLDEKMVPKIADFGLSRFFRGEQSQITKSAIGTHGYVPPEYIDASVLSIKFDIFSLGVVIIKIMTGPTGYFRSAEMSAKQFIELVLANWRKRLPATSVYLLESYSEQVKRCTAIAVSCVEADRSKRPSIGEIVNKLNETETMIQNVARKHTRSSVDQVSEPVTFIGGAVGVLDTLGGVSLNNNHGSGFLGGGTNRGSKVSILAFEVANTIAKASDLWRSCSDESIKELKEEILHSDGVRILISSNSSELLHIAAIGKREELAILSREVIRFGDLFIDPIWHNLRRYFEKSTKDSMPQDHSKEHIGTTVQQLISLAQNTSQVNNQESGLLYISQLELQSEGGSGYFFAFILWLEAYADELVL

SEQ ID NO: 5 - protein, 579 aa, Thinopyrum ponticum
MVFADITKRALCFEYMRNGGLDKCLSDETSGHDWCTRYSIIKGICEGLKYLHEELESPMYHLDLKPANILLDEKMVPKIADFGLSRFFRGEQSQITKSAIGTHGYVPPEYIDASVLSIKFDIFSLGVVIIKIMTGPTGYFRSAEMSAKQFIELVLANWRKRLPATSVYLLESYSEQVKRCTAIAVSCVEADRSKRPSIGEIVNKLNETETMIQNVARKHTRSSVDQVSEPVTFIGGAVGVLDTLGGVSLNNNHGSGFLGGGTNRGSKVSILAFEVANTIAKASDLWRSCSDESIKELKEEILHSDGVRILISSNSSELLHIAAIGKREELAILSREVIRFGDLFIDPIWHNLRRYFEKSTKDSMPQDHSKEHIGTTVQQLISLAQNTSKLYHELHALDRLAQEFQRKFQEEESVPATRRESVMILHSELKRQWKLVKNLKKKSLWSRPLEDIVEKLVDIVSFLDKQIRDAFGEAVPVGTDFIEQVQNKRLGSCGLALHYANIINQIENIVSRPLSLPPSARDNLYHGLPVTVKSALRSRLQSVDTEEEHTVSQIKAEMQKTLRWILPIAENTRRYFKSF

SEQ ID NO: 6 - DNA, 2598 bp, Thinopyrum ponticum
  source 1..2598: mol_type = unassigned DNA
atggataatatatttcgcaaattcggatggaaagctggtagacggcaacaaggagaaaggagagggaatagcacgggcagggcgtcgccaaggcctctttcgtaccatttattaggagaaattacaaatggtttctcggagaaccgaaaacttggtactggtgcatatggaagtgtttacaagggggagcataaagacggggaaaagattgctgtgaagatgcttcatgatactctaggacttgacaatgagcaatttgaaaaggagtattttaaccttgcagatctacagcacaaaaatattgtccgattagttggctattgccatgaaactcggcgagaatgtgtgccttacaacggaagaatggttttcgctgacataacaaagagagcgctttgctttgagtatatgcgtaatggaggtcttgacaaatgtctttcagatgaaactagtggacatgattggtgcacacgctattcaatcattaaggggatttgcgagggtctgaaatacctgcacgaggaattggaatctcctatgtatcatttggacttaaaaccagccaatatattactcgatgagaaaatggtgccgaaaatcgcagatttcggcttgtcaaggttcttcagaggagagcaatcgcaaatcacaaaaagtgctataggaacacatggatacgtaccgcccgaatacatagatgcatctgtactctcaattaagtttgacatattcagcttgggtgttgtaatcataaagataatgactggaccaacaggctacttcagaagtgctgaaatgtctgcaaaacaattcatagaacttgtactggcaaactggaggaagaggctacctgcaacatcagtgtacctgctggagtcatactcggaacaagtaaagaggtgcaccgcaatagctgtaagttgtgtggaggctgatcgatccaaaaggccgtctataggggaaattgtgaataagctgaatgagacagagacaatgattcaaaatgtggcaaggaaacatacacgatcgtcggtggaccaggtatcagaaccagttacatttataggaggagctgttggggtgttagatacccttggcggtgttagcttaaataataatcatggcagtggtttcttgggcggaggaacaaatcgaggaagtaaagtcagtattttggcatttgaagtcgcaaatacaatagctaaagcttccgatttgtggagatcatgctctgacgagagcataaaagaactgaaggaagaaattttgcattcagatggtgtgcgaatattaatttcctcgaattccagtgagctcctgcatattgctgctatcggcaagagggaagaactcgccatcctttcaagagaagtaattcgatttggtgacctgtttatagaccccatatggcataacttgagacgctattttgaaaagtcaacgaaagattccatgccccaggatcattcaaaagagcatatcggaactactgtccagcagttgattagtttggctcaaaacacttctctttaccatgaattgcacgctctggatagattggcgcaggaatttcaaaggaaatttcaggaagaggagtctgtaccagcaactagacgagagagtgttatgattttgcacagtgaactaaagcgccaatggaagcttgtgaaaaatttgaagaagaaatccttgtggtccagacctttggaggatattgtggaaaagcttgttgatatcgtcagttttctagataaacaaatccgggatgcatttggtgaagccgttcctgtaggtactgacttcatcgagcaagttcagaacaaaaggctaggttcctgtggtctggcactacattacgctaacattatcaatcaaattgaaaatatagtttctcggccactctctcttcctcctagtgctagggacaacttgtaccatggactaccagtaacagtgaagtcagctctgcggtcaaggttgcaatcagttgatactgaagaggagcatactgtatctcaaatcaaagctgaaatgcagaaaacccttcgctggattctgccaatagcagaaaatacaaggagagcacaccaagtatttggatgggtcggcgaatgggcacactttgggagtgacatggatgagaagtcaggttcccggcacagcgtgatcggggtccagacgcttcatcatgccgacaaggcgaagacggaggagcacatcctcgagcttgtggtgttgctccatcatctcgtggtccaggtgaaaagccgaggctacgagcacaacaattccacaaagcaggaccggtctcggtcctgcaaggtatcatcggaaccgcaagccgatgccaagcactacacatctccgacgaacaactgtgagatctgcccgagcccgctgtcggactccgagcgccggacgctggaccacctgagcttcaagagctgcgagcctcagcctggcagggtggacaaggcgcataggagcggggactcgtgccggagccatggcagctcgccggcgagggagttcagtcgtagcttgacctcgggccgtgagctggtgaaggatctggatgtaatagatggtctagcagac

SEQ ID NO: 7 - DNA, 2379 bp, Thinopyrum ponticum
  source 1..2379: mol_type = unassigned DNA
atgcttcatgatactctaggacttgacaatgagcaatttgaaaaggagtattttaaccttgcagatctacagcacaaaaatattgtccgattagttggctattgccatgaaactcggcgagaatgtgtgccttacaacggaagaatggttttcgctgacataacaaagagagcgctttgctttgagtatatgcgtaatggaggtcttgacaaatgtctttcagatgaaactagtggacatgattggtgcacacgctattcaatcattaaggggatttgcgagggtctgaaatacctgcacgaggaattggaatctcctatgtatcatttggacttaaaaccagccaatatattactcgatgagaaaatggtgccgaaaatcgcagatttcggcttgtcaaggttcttcagaggagagcaatcgcaaatcacaaaaagtgctataggaacacatggatacgtaccgcccgaatacatagatgcatctgtactctcaattaagtttgacatattcagcttgggtgttgtaatcataaagataatgactggaccaacaggctacttcagaagtgctgaaatgtctgcaaaacaattcatagaacttgtactggcaaactggaggaagaggctacctgcaacatcagtgtacctgctggagtcatactcggaacaagtaaagaggtgcaccgcaatagctgtaagttgtgtggaggctgatcgatccaaaaggccgtctataggggaaattgtgaataagctgaatgagacagagacaatgattcaaaatgtggcaaggaaacatacacgatcgtcggtggaccaggtatcagaaccagttacatttataggaggagctgttggggtgttagatacccttggcggtgttagcttaaataataatcatggcagtggtttcttgggcggaggaacaaatcgaggaagtaaagtcagtattttggcatttgaagtcgcaaatacaatagctaaagcttccgatttgtggagatcatgctctgacgagagcataaaagaactgaaggaagaaattttgcattcagatggtgtgcgaatattaatttcctcgaattccagtgagctcctgcatattgctgctatcggcaagagggaagaactcgccatcctttcaagagaagtaattcgatttggtgacctgtttatagaccccatatggcataacttgagacgctattttgaaaagtcaacgaaagattccatgccccaggatcattcaaaagagcatatcggaactactgtccagcagttgattagtttggctcaaaacacttctctttaccatgaattgcacgctctggatagattggcgcaggaatttcaaaggaaatttcaggaagaggagtctgtaccagcaactagacgagagagtgttatgattttgcacagtgaactaaagcgccaatggaagcttgtgaaaaatttgaagaagaaatccttgtggtccagacctttggaggatattgtggaaaagcttgttgatatcgtcagttttctagataaacaaatccgggatgcatttggtgaagccgttcctgtaggtactgacttcatcgagcaagttcagaacaaaaggctaggttcctgtggtctggcactacattacgctaacattatcaatcaaattgaaaatatagtttctcggccactctctcttcctcctagtgctagggacaacttgtaccatggactaccagtaacagtgaagtcagctctgcggtcaaggttgcaatcagttgatactgaagaggagcatactgtatctcaaatcaaagctgaaatgcagaaaacccttcgctggattctgccaatagcagaaaatacaaggagagcacaccaagtatttggatgggtcggcgaatgggcacactttgggagtgacatggatgagaagtcaggttcccggcacagcgtgatcggggtccagacgcttcatcatgccgacaaggcgaagacggaggagcacatcctcgagcttgtggtgttgctccatcatctcgtggtccaggtgaaaagccgaggctacgagcacaacaattccacaaagcaggaccggtctcggtcctgcaaggtatcatcggaaccgcaagccgatgccaagcactacacatctccgacgaacaactgtgagatctgcccgagcccgctgtcggactccgagcgccggacgctggaccacctgagcttcaagagctgcgagcctcagcctggcagggtggacaaggcgcataggagcggggactcgtgccggagccatggcagctcgccggcgagggagttcagtcgtagcttgacctcgggccgtgagctggtgaaggatctggatgtaatagatggtctagcagac

SEQ ID NO: 8 - DNA, 1434 bp, Thinopyrum ponticum
  source 1..1434: mol_type = other DNA
atgcttcatgatactctaggacttgacaatgagcaatttgaaaaggagtattttaaccttgcagatctacagcacaaaaatattgtccgattagttggctattgccatgaaactcggcgagaatgtgtgccttacaacggaagaatggttttcgctgacataacaaagagagcgctttgctttgagtatatgcgtaatggaggtcttgacaaatgtctttcagatgaaactagtggacatgattggtgcacacgctattcaatcattaaggggatttgcgagggtctgaaatacctgcacgaggaattggaatctcctatgtatcatttggacttaaaaccagccaatatattactcgatgagaaaatggtgccgaaaatcgcagatttcggcttgtcaaggttcttcagaggagagcaatcgcaaatcacaaaaagtgctataggaacacatggatacgtaccgcccgaatacatagatgcatctgtactctcaattaagtttgacatattcagcttgggtgttgtaatcataaagataatgactggaccaacaggctacttcagaagtgctgaaatgtctgcaaaacaattcatagaacttgtactggcaaactggaggaagaggctacctgcaacatcagtgtacctgctggagtcatactcggaacaagtaaagaggtgcaccgcaatagctgtaagttgtgtggaggctgatcgatccaaaaggccgtctataggggaaattgtgaataagctgaatgagacagagacaatgattcaaaatgtggcaaggaaacatacacgatcgtcggtggaccaggtatcagaaccagttacatttataggaggagctgttggggtgttagatacccttggcggtgttagcttaaataataatcatggcagtggtttcttgggcggaggaacaaatcgaggaagtaaagtcagtattttggcatttgaagtcgcaaatacaatagctaaagcttccgatttgtggagatcatgctctgacgagagcataaaagaactgaaggaagaaattttgcattcagatggtgtgcgaatattaatttcctcgaattccagtgagctcctgcatattgctgctatcggcaagagggaagaactcgccatcctttcaagagaagtaattcgatttggtgacctgtttatagaccccatatggcataacttgagacgctattttgaaaagtcaacgaaagattccatgccccaggatcattcaaaagagcatatcggaactactgtccagcagttgattagtttggctcaaaacacttctcaggttaataaccaagaatcaggtctcttgtatatcagccagctagaactacagtctgagggtggatcaggctatttttttgcattcatactctggcttgaagcttatgctgatgagctagttcta

SEQ ID NO: 9 - DNA, 1737 bp, Thinopyrum ponticum
  source 1..1737: mol_type = unassigned DNA
atggttttcgctgacataacaaagagagcgctttgctttgagtatatgcgtaatggaggtcttgacaaatgtctttcagatgaaactagtggacatgattggtgcacacgctattcaatcattaaggggatttgcgagggtctgaaatacctgcacgaggaattggaatctcctatgtatcatttggacttaaaaccagccaatatattactcgatgagaaaatggtgccgaaaatcgcagatttcggcttgtcaaggttcttcagaggagagcaatcgcaaatcacaaaaagtgctataggaacacatggatacgtaccgcccgaatacatagatgcatctgtactctcaattaagtttgacatattcagcttgggtgttgtaatcataaagataatgactggaccaacaggctacttcagaagtgctgaaatgtctgcaaaacaattcatagaacttgtactggcaaactggaggaagaggctacctgcaacatcagtgtacctgctggagtcatactcggaacaagtaaagaggtgcaccgcaatagctgtaagttgtgtggaggctgatcgatccaaaaggccgtctataggggaaattgtgaataagctgaatgagacagagacaatgattcaaaatgtggcaaggaaacatacacgatcgtcggtggaccaggtatcagaaccagttacatttataggaggagctgttggggtgttagatacccttggcggtgttagcttaaataataatcatggcagtggtttcttgggcggaggaacaaatcgaggaagtaaagtcagtattttggcatttgaagtcgcaaatacaatagctaaagcttccgatttgtggagatcatgctctgacgagagcataaaagaactgaaggaagaaattttgcattcagatggtgtgcgaatattaatttcctcgaattccagtgagctcctgcatattgctgctatcggcaagagggaagaactcgccatcctttcaagagaagtaattcgatttggtgacctgtttatagaccccatatggcataacttgagacgctattttgaaaagtcaacgaaagattccatgccccaggatcattcaaaagagcatatcggaactactgtccagcagttgattagtttggctcaaaacacttctaagctttaccatgaattgcacgctctggatagattggcgcaggaatttcaaaggaaatttcaggaagaggagtctgtaccagcaactagacgagagagtgttatgattttgcacagtgaactaaagcgccaatggaagcttgtgaaaaatttgaagaagaaatccttgtggtccagacctttggaggatattgtggaaaagcttgttgatatcgtcagttttctagataaacaaatccgggatgcatttggtgaagccgttcctgtaggtactgacttcatcgagcaagttcagaacaaaaggctaggttcctgtggtctggcactacattacgctaacattatcaatcaaattgaaaatatagtttctcggccactctctcttcctcctagtgctagggacaacttgtaccatggactaccagtaacagtgaagtcagctctgcggtcaaggttgcaatcagttgatactgaagaggagcatactgtatctcaaatcaaagctgaaatgcagaaaacccttcgctggattctgccaatagcagaaaatacaaggaggtacttcaaatcattc

SEQ ID NO: 10 - DNA, 2950 bp, Thinopyrum ponticum
  source 1..2950: mol_type = unassigned DNA
atcagctcatcatcgtcgcaaggagctgtagcccaagaggccaagaccatcacgagctggcggttcattcccgttccaggagcgttgaccagctccacatcgttcctcacagcagcctttttcaaggcatggataatatatttcgcaaattcggatggaaagctggtagacggcaacaaggagaaaggagagggaatagcacgggcagggcgtcgccaaggcctctttcgtaccatttattaggagaaattacaaatggtttctcggagaaccgaaaacttggtactggtgcatatggaagtgtttacaagggggagcataaagacggggaaaagattgctgtgaagatgcttcatgatactctaggacttgacaatgagcaatttgaaaaggagtattttaaccttgcagatctacagcacaaaaatattgtccgattagttggctattgccatgaaactcggcgagaatgtgtgccttacaacggaagaatggttttcgctgacataacaaagagagcgctttgctttgagtatatgcgtaatggaggtcttgacaaatgtctttcagatgaaactagtggacatgattggtgcacacgctattcaatcattaaggggatttgcgagggtctgaaatacctgcacgaggaattggaatctcctatgtatcatttggacttaaaaccagccaatatattactcgatgagaaaatggtgccgaaaatcgcagatttcggcttgtcaaggttcttcagaggagagcaatcgcaaatcacaaaaagtgctataggaacacatggatacgtaccgcccgaatacatagatgcatctgtactctcaattaagtttgacatattcagcttgggtgttgtaatcataaagataatgactggaccaacaggctacttcagaagtgctgaaatgtctgcaaaacaattcatagaacttgtactggcaaactggaggaagaggctacctgcaacatcagtgtacctgctggagtcatactcggaacaagtaaagaggtgcaccgcaatagctgtaagttgtgtggaggctgatcgatccaaaaggccgtctataggggaaattgtgaataagctgaatgagacagagacaatgattcaaaatgtggcaaggaaacatacacgatcgtcggtggaccaggtatcagaaccagttacatttataggaggagctgttggggtgttagatacccttggcggtgttagcttaaataataatcatggcagtggtttcttgggcggaggaacaaatcgaggaagtaaagtcagtattttggcatttgaagtcgcaaatacaatagctaaagcttccgatttgtggagatcatgctctgacgagagcataaaagaactgaaggaagaaattttgcattcagatggtgtgcgaatattaatttcctcgaattccagtgagctcctgcatattgctgctatcggcaagagggaagaactcgccatcctttcaagagaagtaattcgatttggtgacctgtttatagaccccatatggcataacttgagacgctattttgaaaagtcaacgaaagattccatgccccaggatcattcaaaagagcatatcggaactactgtccagcagttgattagtttggctcaaaacacttctctttaccatgaattgcacgctctggatagattggcgcaggaatttcaaaggaaatttcaggaagaggagtctgtaccagcaactagacgagagagtgttatgattttgcacagtgaactaaagcgccaatggaagcttgtgaaaaatttgaagaagaaatccttgtggtccagacctttggaggatattgtggaaaagcttgttgatatcgtcagttttctagataaacaaatccgggatgcatttggtgaagccgttcctgtaggtactgacttcatcgagcaagttcagaacaaaaggctaggttcctgtggtctggcactacattacgctaacattatcaatcaaattgaaaatatagtttctcggccactctctcttcctcctagtgctagggacaacttgtaccatggactaccagtaacagtgaagtcagctctgcggtcaaggttgcaatcagttgatactgaagaggagcatactgtatctcaaatcaaagctgaaatgcagaaaacccttcgctggattctgccaatagcagaaaatacaaggagagcacaccaagtatttggatgggtcggcgaatgggcacactttgggagtgacatggatgagaagtcaggttcccggcacagcgtgatcggggtccagacgcttcatcatgccgacaaggcgaagacggaggagcacatcctcgagcttgtggtgttgctccatcatctcgtggtccaggtgaaaagccgaggctacgagcacaacaattccacaaagcaggaccggtctcggtcctgcaaggtatcatcggaaccgcaagccgatgccaagcactacacatctccgacgaacaactgtgagatctgcccgagcccgctgtcggactccgagcgccggacgctggaccacctgagcttcaagagctgcgagcctcagcctggcagggtggacaaggcgcataggagcggggactcgtgccggagccatggcagctcgccggcgagggagttcagtcgtagcttgacctcgggccgtgagctggtgaaggatctggatgtaatagatggtctagcagactgactatcgtttagttaagagtgaagacatttggttgcacagcagagtaggtattctttggcctttgccgccgccttcttctatctcgtcgatcagatcgtatatatcttgtatgtgtgttttcgaccgttacatatgtagattatataaatgacagtgttacagctgggttgtgatttgaatggaaatatagcaagatcacaagtgatgctccattgcctcca

SEQ ID NO: 11 - DNA, 3046 bp, Thinopyrum ponticum
  source 1..3046: mol_type = unassigned DNA
atcagctcatcatcgtcgcaaggagctgtagcccaagaggccaagaccatcacgagctggcggttcattcccgttccaggagcgttgaccagctccacatcgttcctcacagcagcctttttcaaggcatggataatatatttcgcaaattcggatggaaagctggtagacggcaacaaggagaaaggcagtttgattcgccatgagtctcgattccttatacaggcataaaaatttctctgatgtgacgagtgaaattgctatatttatttttgcctaccaggagagggaatagcacgggcagggcgtcgccaaggcctctttcgtaccatttattaggagaaattacaaatggtttctcggagaaccgaaaacttggtactggtgcatatggaagtgtttacaagggggagcataaagacggggaaaagattgctgtgaagatgcttcatgatactctaggacttgacaatgagcaatttgaaaaggagtattttaaccttgcagatctacagcacaaaaatattgtccgattagttggctattgccatgaaactcggcgagaatgtgtgccttacaacggaagaatggttttcgctgacataacaaagagagcgctttgctttgagtatatgcgtaatggaggtcttgacaaatgtctttcagatgaaactagtggacatgattggtgcacacgctattcaatcattaaggggatttgcgagggtctgaaatacctgcacgaggaattggaatctcctatgtatcatttggacttaaaaccagccaatatattactcgatgagaaaatggtgccgaaaatcgcagatttcggcttgtcaaggttcttcagaggagagcaatcgcaaatcacaaaaagtgctataggaacacatggatacgtaccgcccgaatacatagatgcatctgtactctcaattaagtttgacatattcagcttgggtgttgtaatcataaagataatgactggaccaacaggctacttcagaagtgctgaaatgtctgcaaaacaattcatagaacttgtactggcaaactggaggaagaggctacctgcaacatcagtgtacctgctggagtcatactcggaacaagtaaagaggtgcaccgcaatagctgtaagttgtgtggaggctgatcgatccaaaaggccgtctataggggaaattgtgaataagctgaatgagacagagacaatgattcaaaatgtggcaaggaaacatacacgatcgtcggtggaccaggtatcagaaccagttacatttataggaggagctgttggggtgttagatacccttggcggtgttagcttaaataataatcatggcagtggtttcttgggcggaggaacaaatcgaggaagtaaagtcagtattttggcatttgaagtcgcaaatacaatagctaaagcttccgatttgtggagatcatgctctgacgagagcataaaagaactgaaggaagaaattttgcattcagatggtgtgcgaatattaatttcctcgaattccagtgagctcctgcatattgctgctatcggcaagagggaagaactcgccatcctttcaagagaagtaattcgatttggtgacctgtttatagaccccatatggcataacttgagacgctattttgaaaagtcaacgaaagattccatgccccaggatcattcaaaagagcatatcggaactactgtccagcagttgattagtttggctcaaaacacttctctttaccatgaattgcacgctctggatagattggcgcaggaatttcaaaggaaatttcaggaagaggagtctgtaccagcaactagacgagagagtgttatgattttgcacagtgaactaaagcgccaatggaagcttgtgaaaaatttgaagaagaaatccttgtggtccagacctttggaggatattgtggaaaagcttgttgatatcgtcagttttctagataaacaaatccgggatgcatttggtgaagccgttcctgtaggtactgacttcatcgagcaagttcagaacaaaaggctaggttcctgtggtctggcactacattacgctaacattatcaatcaaattgaaaatatagtttctcggccactctctcttcctcctagtgctagggacaacttgtaccatggactaccagtaacagtgaagtcagctctgcggtcaaggttgcaatcagttgatactgaagaggagcatactgtatctcaaatcaaagctgaaatgcagaaaacccttcgctggattctgccaatagcagaaaatacaaggagagcacaccaagtatttggatgggtcggcgaatgggcacactttgggagtgacatggatgagaagtcaggttcccggcacagcgtgatcggggtccagacgcttcatcatgccgacaaggcgaagacggaggagcacatcctcgagcttgtggtgttgctccatcatctcgtggtccaggtgaaaagccgaggctacgagcacaacaattccacaaagcaggaccggtctcggtcctgcaaggtatcatcggaaccgcaagccgatgccaagcactacacatctccgacgaacaactgtgagatctgcccgagcccgctgtcggactccgagcgccggacgctggaccacctgagcttcaagagctgcgagcctcagcctggcagggtggacaaggcgcataggagcggggactcgtgccggagccatggcagctcgccggcgagggagttcagtcgtagcttgacctcgggccgtgagctggtgaaggatctggatgtaatagatggtctagcagactgactatcgtttagttaagagtgaagacatttggttgcacagcagagtaggtattctttggcctttgccgccgccttcttctatctcgtcgatcagatcgtatatatcttgtatgtgtgttttcgaccgttacatatgtagattatataaatgacagtgttacagctgggttgtgatttgaatggaaatatagcaagatcacaagtgatgctccattgcctcca

SEQ ID NO: 12 - DNA, 3265 bp, Thinopyrum ponticum
  source 1..3265: mol_type = unassigned DNA
atcagctcatcatcgtcgcaaggagctgtagcccaagaggccaagaccatcacgagctggcggttcattcccgttccaggagcgttgaccagctccacatcgttcctcacagcagcctttttcaaggcatggataatatatttcgcaaattcggatggaaagctggtagacggcaacaaggagaaaggcagtttgattcgccatgagtctcgattccttatacaggcataaaaatttctctgatgtgacgagtgaaattgctatatttatttttgcctaccaggagagggaatagcacgggcagggcgtcgccaaggcctctttcgtaccatttattaggagaaattacaaatggtttctcggagaaccgaaaacttggtactggtgcatatggaagtgtttacaagggggagcataaagacggggaaaagattgctgtgaagatgcttcatgatactctaggacttgacaatgagcaatttgaaaaggagtattttaaccttgcagatctacagcacaaaaatattgtccgattagttggctattgccatgaaactcggcgagaatgtgtgccttacaacggaagaatggttttcgctgacataacaaagagagcgctttgctttgagtatatgcgtaatggaggtcttgacaaatgtctttcagatgaaactagtggacatgattggtgcacacgctattcaatcattaaggggatttgcgagggtctgaaatacctgcacgaggaattggaatctcctatgtatcatttggacttaaaaccagccaatatattactcgatgagaaaatggtgccgaaaatcgcagatttcggcttgtcaaggttcttcagaggagagcaatcgcaaatcacaaaaagtgctataggaacacatggatacgtaccgcccgaatacatagatgcatctgtactctcaattaagtttgacatattcagcttgggtgttgtaatcataaagataatgactggaccaacaggctacttcagaagtgctgaaatgtctgcaaaacaattcatagaacttgtactggcaaactggaggaagaggctacctgcaacatcagtgtacctgctggagtcatactcggaacaagtaaagaggtgcaccgcaatagctgtaagttgtgtggaggctgatcgatccaaaaggccgtctataggggaaattgtgaataagctgaatgagacagagacaatgattcaaaatgtggcaaggaaacatacacgatcgtcggtggaccaggtatcagaaccagttacatttataggaggagctgttggggtgttagatacccttggcggtgttagcttaaataataatcatggcagtggtttcttgggcggaggaacaaatcgaggaagtaaagtcagtattttggcatttgaagtcgcaaatacaatagctaaagcttccgatttgtggagatcatgctctgacgagagcataaaagaactgaaggaagaaattttgcattcagatggtgtgcgaatattaatttcctcgaattccagtgagctcctgcatattgctgctatcggcaagagggaagaactcgccatcctttcaagagaagtaattcgatttggtgacctgtttatagaccccatatggcataacttgagacgctattttgaaaagtcaacgaaagattccatgccccaggatcattcaaaagagcatatcggaactactgtccagcagttgattagtttggctcaaaacacttctcaggttaataaccaagaatcaggtctcttgtatatcagccagctagaactacagtctgagggtggatcaggctatttttttgcattcatactctggcttgaagcttatgctgatgagctagttctataactgcagaagctttaccatgaattgcacgctctggatagattggcgcaggaatttcaaaggaaatttcaggaagaggagtctgtaccagcaactagacgagagagtgttatgattttgcacagtgaactaaagcgccaatggaagcttgtgaaaaatttgaagaagaaatccttgtggtccagacctttggaggatattgtggaaaagcttgttgatatcgtcagttttctagataaacaaatccgggatgcatttggtgaagccgttcctgtaggtactgacttcatcgagcaagttcagaacaaaaggctaggttcctgtggtctggcactacattacgctaacattatcaatcaaattgaaaatatagtttctcggccactctctcttcctcctagtgctagggacaacttgtaccatggactaccagtaacagtgaagtcagctctgcggtcaaggttgcaatcagttgatactgaagaggagcatactgtatctcaaatcaaagctgaaatgcagaaaacccttcgctggattctgccaatagcagaaaatacaaggaggtacttcaaatcattctgatttctgagcatagttgtttttctccttgtttacaaaattcactgtttccatacttgtacaagagcacaccaagtatttggatgggtcggcgaatgggcacactttgggagtgacatggatgagaagtcaggttcccggcacagcgtgatcggggtccagacgcttcatcatgccgacaaggcgaagacggaggagcacatcctcgagcttgtggtgttgctccatcatctcgtggtccaggtgaaaagccgaggctacgagcacaacaattccacaaagcaggaccggtctcggtcctgcaaggtatcatcggaaccgcaagccgatgccaagcactacacatctccgacgaacaactgtgagatctgcccgagcccgctgtcggactccgagcgccggacgctggaccacctgagcttcaagagctgcgagcctcagcctggcagggtggacaaggcgcataggagcggggactcgtgccggagccatggcagctcgccggcgagggagttcagtcgtagcttgacctcgggccgtgagctggtgaaggatctggatgtaatagatggtctagcagactgactatcgtttagttaagagtgaagacatttggttgcacagcagagtaggtattctttggcctttgccgccgccttcttctatctcgtcgatcagatcgtatatatcttgtatgtgtgttttcgaccgttacatatgtagattatataaatgacagtgttacagctgggttgtgatttgaatggaaatatagcaagatcacaagtgatgctccattgcctcca

SEQ ID NO: 13 - DNA, 2934 bp, Thinopyrum ponticum
  source 1..2934: mol_type = unassigned DNA
atcagctcatcatcgtcgcaaggagctgtagcccaagaggccaagaccatcacgagctggcggttcattcccgttccaggagcgttgaccagctccacatcgttcctcacagcagcctttttcaaggcatggataatatatttcgcaaattcggatggaaagctggtagacggcaacaaggagaaaggagagggaatagcacgggcagggcgtcgccaaggcctctttcgtaccatttattaggagaaattacaaatggtttctcggagaaccgaaaacttggtactggtgcatatggaagtgtttacaagatctacagcacaaaaatattgtccgattagttggctattgccatgaaactcggcgagaatgtgtgccttacaacggaagaatggttttcgctgacataacaaagagagcgctttgctttgagtatatgcgtaatggaggtcttgacaaatgtctttcagatgaaactagtggacatgattggtgcacacgctattcaatcattaaggggatttgcgagggtctgaaatacctgcacgaggaattggaatctcctatgtatcatttggacttaaaaccagccaatatattactcgatgagaaaatggtgccgaaaatcgcagatttcggcttgtcaaggttcttcagaggagagcaatcgcaaatcacaaaaagtgctataggaacacatggatacgtaccgcccgaatacatagatgcatctgtactctcaattaagtttgacatattcagcttgggtgttgtaatcataaagataatgactggaccaacaggctacttcagaagtgctgaaatgtctgcaaaacaattcatagaacttgtactggcaaactggaggaagaggctacctgcaacatcagtgtacctgctggagtcatactcggaacaagtaaagaggtgcaccgcaatagctgtaagttgtgtggaggctgatcgatccaaaaggccgtctataggggaaattgtgaataagctgaatgagacagagacaatgattcaaaatgtggcaaggaaacatacacgatcgtcggtggaccaggtatcagaaccagttacatttataggaggagctgttggggtgttagatacccttggcggtgttagcttaaataataatcatggcagtggtttcttgggcggaggaacaaatcgaggaagtaaagtcagtattttggcatttgaagtcgcaaatacaatagctaaagcttccgatttgtggagatcatgctctgacgagagcataaaagaactgaaggaagaaattttgcattcagatggtgtgcgaatattaatttcctcgaattccagtgagctcctgcatattgctgctatcggcaagagggaagaactcgccatcctttcaagagaagtaattcgatttggtgacctgtttatagaccccatatggcataacttgagacgctattttgaaaagtcaacgaaagattccatgccccaggatcattcaaaagagcatatcggaactactgtccagcagttgattagtttggctcaaaacacttctaagctttaccatgaattgcacgctctggatagattggcgcaggaatttcaaaggaaatttcaggaagaggagtctgtaccagcaactagacgagagagtgttatgattttgcacagtgaactaaagcgccaatggaagcttgtgaaaaatttgaagaagaaatccttgtggtccagacctttggaggatattgtggaaaagcttgttgatatcgtcagttttctagataaacaaatccgggatgcatttggtgaagccgttcctgtaggtactgacttcatcgagcaagttcagaacaaaaggctaggttcctgtggtctggcactacattacgctaacattatcaatcaaattgaaaatatagtttctcggccactctctcttcctcctagtgctagggacaacttgtaccatggactaccagtaacagtgaagtcagctctgcggtcaaggttgcaatcagttgatactgaagaggagcatactgtatctcaaatcaaagctgaaatgcagaaaacccttcgctggattctgccaatagcagaaaatacaaggaggtacttcaaatcattctgatttctgagcatagttgtttttctccttgtttacaaaattcactgtttccatacttgtacaagagcacaccaagtatttggatgggtcggcgaatgggcacactttgggagtgacatggatgagaagtcaggttcccggcacagcgtgatcggggtccagacgcttcatcatgccgacaaggcgaagacggaggagcacatcctcgagcttgtggtgttgctccatcatctcgtggtccaggtgaaaagccgaggctacgagcacaacaattccacaaagcaggaccggtctcggtcctgcaaggtatcatcggaaccgcaagccgatgccaagcactacacatctccgacgaacaactgtgagatctgcccgagcccgctgtcggactccgagcgccggacgctggaccacctgagcttcaagagctgcgagcctcagcctggcagggtggacaaggcgcataggagcggggactcgtgccggagccatggcagctcgccggcgagggagttcagtcgtagcttgacctcgggccgtgagctggtgaaggatctggatgtaatagatggtctagcagactgactatcgtttagttaagagtgaagacatttggttgcacagcagagtaggtattctttggcctttgccgccgccttcttctatctcgtcgatcagatcgtatatatcttgtatgtgtgttttcgaccgttacatatgtagattatataaatgacagtgttacagctgggttgtgatttgaatggaaatatagcaagatcacaagtgatgctccattgcctcca

SEQ ID NO: 14 - DNA, 11096 bp, Aegilops sharonensis
  source 1..11096: mol_type = genomic DNA
ggtggtgattagataacggtaatgactatgatgattaaatagtgataatgacgactacgatgttttttagctagctagctagagtatgttgatgatatgatgcaagagtttttatattaatatgataatgatgatgagttattatatcatttaatgaaagaaaccatatattagtttcagctggatggatcctacctaagtgatcaagtatatatatatgccatatccacatatattatacttgatcacctaattaggtgatcaagtataatatcgatatggcatatacttgatcacttagctagctagatccttcagtcgaaactaatccgcggtactttcatctaatgatttagcaactcattataatgtaaaagaatcactaaattaaattaaaaacacaaaactaaagcaaaaataaaaaaaaacacccaaacatttagtactggttggtgttaccaaccggtactaaagccctacgcgcacacgggtctggctcgtgccacgtgtttgcactttagcgccggtttgtgacgaaccggtactaaagggggggggggctttagtccccactctttagtgccggttggcgaaccggcactaaaggccggttctgcactagtgactcataaacaggtgaagcacaatcccttacaaaactgttcttctgggtctggtactttgggattgaaccataccctttccttcaacttcatattccttctcttcctaccacttggtagtttataggttccttcgggctcatcttcttcatcactaatgccataatcactaggaaaacatttttcatcagcttcaggcaaccaatctagtttttccattttctcagcctcactgtgtgatctaattgttggaccaggatttctcactgccttcagtttttgtgccattggtctatcttcatcatcagaggaagaaactgcttgcagttccacattgttgccatctgaatcagatagaaattctgatacatcagtgtcaccttcaaaatgattgaggtcagcttctctctcaagaatacttttcgtaagctcttcctttctgtccatgcttgtatccaccaacttagtgcaactttgttggatgttaatgcaatgatcaaccaaataataatctccgttttcatctacattacatagctcattttccctcacaactcttatgttcacactcttttcatttagagagtggagcaacatctgctgcacatcatcttcagaagatattttctctaagccttctactccctttatgatcatcactaacatagtacatataatcatcagaacaacaaccctcactcctaataagagataccagagtcccaaatgatatgtctgactggtacatgtttctaacagcagaatctctgccttcgaaatggaaccaaattccccattttgggtcatcaatactggacaaaaagggaatgaattattataaagaatgcacattgttaaagttcctaacaatttgcaattactctgttatcatacattttgggaaactgacctaaaatacatcctgtcaataaaataaaactgaaaaagaagaagatatgttgtacctgccgcctcccgcatgaggaagcttgtgccggccgcgaggtgcacttcttgtcgtcgtcggtggtggagccgtcgccgccgccaactccagattcaggcaggtgcgccacaaagctagagctgcctagccaggtatcgacctccgacaggtcgacgcggcccccaacaacgtcaggaggttggctgctgaacgaattgccgccggagtttggatccacaaccgccattaccaccgccgcctagggatacagaggagagagctcggtggagagagatgtggttcggccggtcggtcggccgttttgaccagccctgcctggttccgaccgagctgggccactaacgggcgcgagcgggcgccatttgtcgttagggcagccgtcggcctgccatgtgggccagccctgtcaggtaagcggttaaactggctaaaacacgatcaacacgacttggtagttttttgtcacaaaatataaaattttcggtagttatcagtctttttttgaaaatgatagttctatgggacggtaacccctaatatggtagttttttgtcaaatacttcacggaccagtcggtgttttgcattggtagatttgcttggatccaatcttgaaagaactagcgtgacgagcaaaaagacgacaatgcgtcaccgatttgtgagatgaagttttttttttgacaactgtttgtgagatgaagcttattaaactgaaaaaaaatacagtctgcccccagcacgcgtctttcaaaattcaggctgactcggtcatcaggccggggtttgcttaataattgacacttgggcgactggtcttctgtacagctagcaagcatatgcgcggtcactccagacgaccaccagatctggatctctttattcccctttgggctttggtcaacgcttttgcttggtgagcaccatcgatcggctgcaggctgctcatggtctactagcagcttggcgagtctccgtgtccccttccctcctttccccaggtgagtactaattaggatgctgaatgctatagctgcttccctcttgtctcgatttcggaactccagctatctgagcacctgtgtatctgctcctcgatttgctttctgcagcaagtcaagggaggaagaagggaaggaaggagaagctgctagcgcctagcggccgaagccttatcgcttgctgacacggaaatggacggatacgagttcgagagggcggagatagatgcactggagcgcgtcgtacgggatccaagagaaaagccaataagcctgtcgttttggcttctcaggcgcataacgaatgatttctctaacgcatctgaaattggtcgaggtggattttcgaacgtttacctggtatgtacgattaaactcatattcccgctaggtgcatgtcaagcaatacctatcttgttaacactagccacaatgagtagtaacatagagtagtaatattgcccacgttactattctatgttactacctctatagtgagaagtgtggtaacatgcaacactttatttattagactatagactcatcttgccttgatatatgtgatgttactcatattagtagtaactagctatgttaccacatgcctctctttctttatttattacttgtcatatcatctattttatctaaatatatattatgttactacttatgttactttcattgtggatagtctaaaaataataacttctaactgtgatgatcaaatacatagggggtgcttccaagtgggttgcgtattgctgtcaaaaagctttaccaacttactgcattggctaatgaatttgaagatgaagttcttaccacaatgagggttgctcacaaaaacatagtgcgactcataggctactgtcgtcacacgcaatctgaagtcttcgaattaaaaggagaatctattttcgcacaggtcagagaaaggttgatctgtacagagtatgtgcctaatggaactcttgatggacatatcactggtatcgacaacatgccatactcaccctactactccctccgttcggaaaaaccttttccttaaatggatgtatctagcatcaagttagtgctagatacatccatttaagaaaaaaatttggaataaatttttttggacggaaggagtattaatttcccctctttatttcttgtttcctttcgtgtatatacatggctgttttctatgcttcaaacatgagttcatggatccacatataaaaggggctgtccgtcacaggttaaagtcactcgaattccgtccatccctctaaccccacttctctagcatcagcgacccacccacgcatctgcttccattgacctccgtctcgcctccgctggtgacaccatgaccagccacactgtccccatcaaatacaagatctcatggaattgtgatgtgtacatgacccagcggttgaataggaatttctggatgatacatagtttcatagtatcttataatgatttaactggggttgtctttgttttgtactctgcttaataattaataagaatggttgtgtgcatcacaaaaatgctgcagccgggggtttgaaccttattttccaagagaagacataattttgtgttcctctatctgtaaagcatttcagaatgacaagatcagctccatgtcatgtttgtacaatacattttcaattttctgcagataagtttcatggaggacttgattgggaccagcgttatcaagttctcaaaggaatttgtcacggtttgcattatctgcacgatgaaatacgcgttatccacagagatatcaaaacagaaaatatattgctagatgataacggcgtgccaaaagtttttgactttggtttttcgaagtgccttcctgatggagaatctttaattaaagatgaagcaacccagggaaccctgtaagctaacgctctttgtaattttcttttcttttgttttgacagtaggtattgcgacatccagatacagtagaattttgtctgccaaatgccttagatctaacatacgttttgtcatgcagagggtatttggcgccggagtcagttgataagcaagaatattcctttaagtccgacatatacagctttggtgttgtgatcatacaattattgactggatcgtggatcagtaagaaccgaaatgttgagctagtaagaactttattgatcacatctgaatgtatctagtgctagctttctttctcgtgagttacaaaatcgatgtgagaaagtggtgctgatttcaggaacctaacattgtgactgtctcaacaattccataactttctaacagaaaaacataggatccagatagacacaatccattctacggtttctcgattctagtgggcctaagtgacagccctggccatccacaacgcgcaacttacttattttcctggcttgttctaaaacccccttattttcctggcttgagccactgaaacatgacactatttttgattctacaaatttgtttattttgtacagaacaagaacaaatatgatcatactttagcacagaaatattgacgaaccttttaaaaaagtgctcaacttttagagtgagaaaggaggagagaaggatatcgaaatggatagagaaatcggatttgggtgtacactggatggagattgtggactaaaagaaatgtgtacaacctccagataactgcagttatgtgtctacattgaattaacttcttgcagtaagtactcatttcccttctggctcttgtatatggtgtaaatctgtacaggaacttaaggagttgagaaaaaggttggtaagagaaggagcatttccatcatgggaaaacaaataccaccaagttagaacatgtctggagattgtgtataactgcatggacatcgacccagataaaaggccgactgcattggaaattatccaacggcttgatgaaaccgaaagcgcgaactattctgttagcagtggccatgcaacaccactttggcaggtacgacaaattgaaagaagcataaaatttcttgagaatcagtttctacaaggccaagacctagcacacacaatttttaagagaatgctgacatcacactgtcactgtatagatgctttcaaattgatttgtatatacacacgcagtcaggagatgaagaatccgactcagcggaagtggatgcttttgagccagagagaacaccatcaaaagacattctttcagatgatgaggaacccgcatcagcagacgcaaatggagaaacgagcacacaaaaacataatatgcctgatcttgtaagtaaacagtcagtgtcggcggaactgtcctcgctagatttccttgagaaaatcacagatggtttttcatacgagcaaatactcgggaaggaaagtctggacgcagatgttcataaagcatttctttatgaggtacgcagagttcaatatttgcttcgatgcgctactgatgcatcccttggtttctacttattcttcgatcttaattatgtagacacatacgctaaatatatgtatatatgtaaacagggcagcatttcaggtccagcaaaggtagttgtgaagaggttaattggagtagcaattccagttgcgaagtttaagagggatgcagaacagctgatgagcctggatcacaagaatatagtaaagctccttggctactgccacgacgaatctagagggcataagctggtacagtttaaagcaaaaccaccgcaagattttaaaggagccgagcaacttctctgctatgaatatatgcacaatggaagcctccgtgagtatcttactggtaatgataccgactccgatcacaaatcagttgctgaataactgcccttccatggaagggagcatatggcaacttccaatacaaatcatacatgatactattcgatccatttgtagcttgctaagctagctccttgatacttctgcctgtctacttgttgtttcataggacaaggatctcgtgaagttgattggcacatgtgctacaaattgatcaaagggacctgcgaaggtttacgctacctccatgagggttgtggagatcgtccaattcttcatttgaatctaaacccgtcaaatatactgctggacgaaaacaacataccacgcatcacgggttttgatttttcgaagctcattggtgaaaagaacacaaaatccgtggttcttaagaagaatggaccactgtacgtaggctctcactgctaccagtttatatttagccctcagttcatcatgcaatgtttatgctttgcaggcttgggactggtcgattttagctagctcgttaccgaaaaaggctttcgccccgctttatatataaagcaacgatcaacagcactaggtacaaacgcacacccccacaacacacgcacacacgcacccaaggcggaatacataggcgttgagcgcagcaacaccaacccaaacactacacgaagaggtgaagccgcatatgacaaaccgtgggctccaaggcggcgccttcaggaaggggacgacaccggagcgccgccactgcctgatccgaggatcagagttttccctggagcaacacgacgagcaatgagagccgtggtgacgccttcaagaagggaatgagtttcgccgccgctggtccgtccgaagataaagcaggttttcaccccggccaccactcaccgtcaccgaacgccacaccctagctaccacgccgcccacacaagccgtggcaaccgggcagcaccaagccacgggctctgcccatgagcaccacggaaccatcaccagggccgccgccccggcatcccagaccttgacaccacctcacccgagatccgccgctaccccaaccaaagaaccacgggcggccccagtgccaagacccaataggccggccaaaactagcctccatcgacccgttttagctagctcgtttaaaggctgttacgttttaattgaagttttgaactgatatgtttggatgcatctcctttctgtatttttttcagaggttacctgccaccggacttcctgtactcaaagggtactgacctcaagtatctacctacggtggacatatacagcttgggtcttataattttagagattgcaacacgtcaagagatcaagggagaccatggaataattattaggactgtaagagaattttctcaaaccttttttttttttttgcggttgaaatttgctcagacctatgatcatgcaatacttatggtttggttaacgacaaataaccgaaaaaattcatccttcatctatatgtgtacataaatagcaaaatgttttgtactctgtgagtatgtattctcactcagatatcacacatgttttgtacacttgtacaccctgtacacttgtaaactaatataagattttttagatcactacttagcaccaacggatcttatattagtttacagagggagtacttctttaccactctgaccatacttcgctaatactccctccgtctcataatagtgtcaaaaaacgtcttacattatggacggagggagtagtttgtagatactctgaaatgagttcaatgaaaattttcaatgatagtctatataaaacatagtttgtgtatatgctgcatatatgtacatacaaatattatattgcaaaaaataaaggtctacatgatattccttttataatgttcacactgggctatcataggataagtaattaagtatattgaaaataataaagagagataatatatccgcttaagtggtatattagaatgtggatcagcacatactcttagcacttgatggtatataatactccctctgtaatggagtagtgtcaaaaaaaaaacatcttgtattaagttatggagggagtaggtggcattattttgtatttaaatatttgctctgatatattatgtaggtcgagacaaactggaggcaagatacacaaatagcaacactgtatggttcactagaagataaattacgggagcaagtaaaaatgtgcattgacattggcctagactgtgtcaagtcaaaccctaaaaagagacctacagctggggccatcatgctctggcttgacaaagggagcaaaccgggagttgccagacctccactccgcgataatgtcacccatgcagatgatcacatccaaggtattctgatcccacaattcttaagtttcgattccttcttttacatgtaattagtactatgctcaaacgaaaacaaaattatgaaaaatagaagtgtagtgccttcatctacttttaagtcgattggtaatacaggcacgatgattattacacacgccatttagtctttgtttttctcaaaggtgaaccacagttccgatctggcgaagctaatagatagtatatgttgatgatctcacgggttttgtaacatttatatgttgtcagaaaaggagaagcagacgccattgaggagatttttcagaagaaagtaagctcaagttactaaaggcatgcatgaatagacagagtctctctcgttcatttgcgagaaagctgcgatggcgaaagtttcttccagatgatcacttgcagctggcgtcagtggtgccattcaggctgctggacacatcgggtgctctgttttccgctcttagttgtcagtgctctgtttttcctctgttttagagttgtaactttcaaggggctggagaaacattcatttgtccactgaactggtgcaaccttattatgaagaggtctgcttcggtacacacacacacacacacacccccgcgcttaacgtataagggcatttttggcatacggtgcttaacgtatcccgcgccgtatgcggcgtacccatacaggtagagccgagcgcgcggctcgagccgatacggctggctcgcaactaccctctcgccgtcgcgcgaaaaaaaaacccacgacctgcgctgatcacgcggcccattccaacgtcgccccgccccgctgatcgccgcccttacccgccgatagccgcctttccttacgctaccaattccaacgtcctcccgttcggccgatcgccgccttctccccgtcttcacccgggatctggccggcgcggacagatcttcgagtcgcccgccgtacgtgatgtcgcctccgtgcaggtcgtcgcggtgacgtggcctacgtggacgccgtcgccgcccgcaactccgtgatcaacctctccggcaacgtcctcgccgtccgcagccacgccaggcgggccgaagtgcacgtgcatgccatgtaagccttctagttcgtcaaaaaaattgaatctgtaaataattatgtgcaatctcttgttagataagaatttctcatctgtatgatcgtagtacatttaatttgatttaaaattgatagatcaatttttattataatattgcagtgtaggttgagagatcatttaatggataaagaaactggatttacggatctgtttttggacacgagtgagtgggatggtagcgatagtgttgatcgtacgaatggtaatgatagtaaagatgatgatgatggcagcgacaatgaatcctggtcgtcgtacgataatttacctgaggaggattttgaaaaggaggagggtgcttgtagtgaaaacgtaagtgtcttgtacattctttttgtcggatgaagaaccatatggtacttacacgtgcatatgatttttttatgtgcaggaggatattgacgttcacaacgaggaaaatcatggtgttgaatctttcggggataacataagccaggttcggtccaaaatttaagatgtcattgaataataaatagttggtaatgacaacttacacttgcttatgtggaatattgtaggctaactttgatggttggagtggtgaagatggggatgagcatgaggatggagctaatatggacattgaggaagctgaaatccagcaacgtgcgctggagacacagttgaaggttatgggtatgacatttgcatcacaatgggaggctcacatgttctataataactacgccaaagaccgtggattcagtatcagaaaagataaggtgaaacgaggaaaaggactttcaaccactgttcggtataggagatacgtttgctcgagggcaggaaagcgtcgcagtaaatttttaaacccggagggccgtacccgcaggctaagaccagagactcgttgcgagtgtggcgcacatttagtcgtgaagttggacaaaggacgtggagtttggttcgtggcagcttttatggatgatcacaaccatttgttggctagaccagatgaggtggtatttctgcggtcacatcgagtgatgggagatcaccagatagctgagatcttggcgatggaaggagctgggatcagaaagcatatcatcgttgacaacttcattagcaggtatggctcgtatgataagtgtgggtttctgagacgagacgtgtacaacctatgttgccgagaaaaaatgaagttgattgcgaagggtgatgctaacacg

SEQ ID NO: 15 - protein, 740 aa, Aegilops sharonensis
MDGYEFERAEIDALERVVRDPREKPISLSFWLLRRITNDFSNASEIGRGGFSNVYLGVLPSGLRIAVKKLYQLTALANEFEDEVLTTMRVAHKNIVRLIGYCRHTQSEVFELKGESIFAQVRERLICTEYVPNGTLDGHITDKFHGGLDWDQRYQVLKGICHGLHYLHDEIRVIHRDIKTENILLDDNGVPKVFDFGFSKCLPDGESLIKDEATQGTLGYLAPESVDKQEYSFKSDIYSFGVVIIQLLTGSWISKNRNVELELKELRKRLVREGAFPSWENKYHQVRTCLEIVYNCMDIDPDKRPTALEIIQRLDETESANYSVSSGHATPLWQSGDEESDSAEVDAFEPERTPSKDILSDDEEPASADANGETSTQKHNMPDLVSKQSVSAELSSLDFLEKITDGFSYEQILGKESLDADVHKAFLYEGSISGPAKVVVKRLIGVAIPVAKFKRDAEQLMSLDHKNIVKLLGYCHDESRGHKLVQFKAKPPQDFKGAEQLLCYEYMHNGSLREYLTGQGSREVDWHMCYKLIKGTCEGLRYLHEGCGDRPILHLNLNPSNILLDENNIPRITGFDFSKLIGEKNTKSVVLKKNGPLGYLPPDFLYSKGTDLKYLPTVDIYSLGLIILEIATRQEIKGDHGIIIRTVETNWRQDTQIATLYGSLEDKLREQVKMCIDIGLDCVKSNPKKRPTAGAIMLWLDKGSKPGVARPPLRDNVTHADDHIQEKEKQTPLRRFFRRK

SEQ ID NO: 16 - DNA, 2220 bp, Aegilops sharonensis
  source 1..2220: mol_type = unassigned DNA
atggacggatacgagttcgagagggcggagatagatgcactggagcgcgtcgtacgggatccaagagaaaagccaataagcctgtcgttttggcttctcaggcgcataacgaatgatttctctaacgcatctgaaattggtcgaggtggattttcgaacgtttacctgggggtgcttccaagtgggttgcgtattgctgtcaaaaagctttaccaacttactgcattggctaatgaatttgaagatgaagttcttaccacaatgagggttgctcacaaaaacatagtgcgactcataggctactgtcgtcacacgcaatctgaagtcttcgaattaaaaggagaatctattttcgcacaggtcagagaaaggttgatctgtacagagtatgtgcctaatggaactcttgatggacatatcactgataagtttcatggaggacttgattgggaccagcgttatcaagttctcaaaggaatttgtcacggtttgcattatctgcacgatgaaatacgcgttatccacagagatatcaaaacagaaaatatattgctagatgataacggcgtgccaaaagtttttgactttggtttttcgaagtgccttcctgatggagaatctttaattaaagatgaagcaacccagggaaccctagggtatttggcgccggagtcagttgataagcaagaatattcctttaagtccgacatatacagctttggtgttgtgatcatacaattattgactggatcgtggatcagtaagaaccgaaatgttgagctagaacttaaggagttgagaaaaaggttggtaagagaaggagcatttccatcatgggaaaacaaataccaccaagttagaacatgtctggagattgtgtataactgcatggacatcgacccagataaaaggccgactgcattggaaattatccaacggcttgatgaaaccgaaagcgcgaactattctgttagcagtggccatgcaacaccactttggcagtcaggagatgaagaatccgactcagcggaagtggatgcttttgagccagagagaacaccatcaaaagacattctttcagatgatgaggaacccgcatcagcagacgcaaatggagaaacgagcacacaaaaacataatatgcctgatcttgtaagtaaacagtcagtgtcggcggaactgtcctcgctagatttccttgagaaaatcacagatggtttttcatacgagcaaatactcgggaaggaaagtctggacgcagatgttcataaagcatttctttatgagggcagcatttcaggtccagcaaaggtagttgtgaagaggttaattggagtagcaattccagttgcgaagtttaagagggatgcagaacagctgatgagcctggatcacaagaatatagtaaagctccttggctactgccacgacgaatctagagggcataagctggtacagtttaaagcaaaaccaccgcaagattttaaaggagccgagcaacttctctgctatgaatatatgcacaatggaagcctccgtgagtatcttactggacaaggatctcgtgaagttgattggcacatgtgctacaaattgatcaaagggacctgcgaaggtttacgctacctccatgagggttgtggagatcgtccaattcttcatttgaatctaaacccgtcaaatatactgctggacgaaaacaacataccacgcatcacgggttttgatttttcgaagctcattggtgaaaagaacacaaaatccgtggttcttaagaagaatggaccactaggttacctgccaccggacttcctgtactcaaagggtactgacctcaagtatctacctacggtggacatatacagcttgggtcttataattttagagattgcaacacgtcaagagatcaagggagaccatggaataattattaggactgtcgagacaaactggaggcaagatacacaaatagcaacactgtatggttcactagaagataaattacgggagcaagtaaaaatgtgcattgacattggcctagactgtgtcaagtcaaaccctaaaaagagacctacagctggggccatcatgctctggcttgacaaagggagcaaaccgggagttgccagacctccactccgcgataatgtcacccatgcagatgatcacatccaagaaaaggagaagcagacgccattgaggagatttttcagaagaaag

SEQ ID NO: 17 - DNA, 2847 bp, Aegilops sharonensis
  source 1..2847: mol_type = unassigned DNA
cagcacgcgtctttcaaaattcaggctgactcggtcatcaggccggggtttgcttaataattgacacttgggcgactggtcttctgtacagctagcaagcatatgcgcggtcactccagacgaccaccagatctggatctctttattcccctttgggctttggtcaacgcttttgcttggtgagcaccatcgatcggctgcaggctgctcatggtctactagcagcttggcgagtctccgtgtccccttccctcctttccccagcaagtcaagggaggaagaagggaaggaaggagaagctgctagcgcctagcggccgaagccttatcgcttgctgacacggaaatggacggatacgagttcgagagggcggagatagatgcactggagcgcgtcgtacgggatccaagagaaaagccaataagcctgtcgttttggcttctcaggcgcataacgaatgatttctctaacgcatctgaaattggtcgaggtggattttcgaacgtttacctgggggtgcttccaagtgggttgcgtattgctgtcaaaaagctttaccaacttactgcattggctaatgaatttgaagatgaagttcttaccacaatgagggttgctcacaaaaacatagtgcgactcataggctactgtcgtcacacgcaatctgaagtcttcgaattaaaaggagaatctattttcgcacaggtcagagaaaggttgatctgtacagagtatgtgcctaatggaactcttgatggacatatcactgataagtttcatggaggacttgattgggaccagcgttatcaagttctcaaaggaatttgtcacggtttgcattatctgcacgatgaaatacgcgttatccacagagatatcaaaacagaaaatatattgctagatgataacggcgtgccaaaagtttttgactttggtttttcgaagtgccttcctgatggagaatctttaattaaagatgaagcaacccagggaaccctagggtatttggcgccggagtcagttgataagcaagaatattcctttaagtccgacatatacagctttggtgttgtgatcatacaattattgactggatcgtggatcagtaagaaccgaaatgttgagctagaacttaaggagttgagaaaaaggttggtaagagaaggagcatttccatcatgggaaaacaaataccaccaagttagaacatgtctggagattgtgtataactgcatggacatcgacccagataaaaggccgactgcattggaaattatccaacggcttgatgaaaccgaaagcgcgaactattctgttagcagtggccatgcaacaccactttggcagtcaggagatgaagaatccgactcagcggaagtggatgcttttgagccagagagaacaccatcaaaagacattctttcagatgatgaggaacccgcatcagcagacgcaaatggagaaacgagcacacaaaaacataatatgcctgatcttgtaagtaaacagtcagtgtcggcggaactgtcctcgctagatttccttgagaaaatcacagatggtttttcatacgagcaaatactcgggaaggaaagtctggacgcagatgttcataaagcatttctttatgagggcagcatttcaggtccagcaaaggtagttgtgaagaggttaattggagtagcaattccagttgcgaagtttaagagggatgcagaacagctgatgagcctggatcacaagaatatagtaaagctccttggctactgccacgacgaatctagagggcataagctggtacagtttaaagcaaaaccaccgcaagattttaaaggagccgagcaacttctctgctatgaatatatgcacaatggaagcctccgtgagtatcttactggacaaggatctcgtgaagttgattggcacatgtgctacaaattgatcaaagggacctgcgaaggtttacgctacctccatgagggttgtggagatcgtccaattcttcatttgaatctaaacccgtcaaatatactgctggacgaaaacaacataccacgcatcacgggttttgatttttcgaagctcattggtgaaaagaacacaaaatccgtggttcttaagaagaatggaccactaggttacctgccaccggacttcctgtactcaaagggtactgacctcaagtatctacctacggtggacatatacagcttgggtcttataattttagagattgcaacacgtcaagagatcaagggagaccatggaataattattaggactgtcgagacaaactggaggcaagatacacaaatagcaacactgtatggttcactagaagataaattacgggagcaagtaaaaatgtgcattgacattggcctagactgtgtcaagtcaaaccctaaaaagagacctacagctggggccatcatgctctggcttgacaaagggagcaaaccgggagttgccagacctccactccgcgataatgtcacccatgcagatgatcacatccaagaaaaggagaagcagacgccattgaggagatttttcagaagaaagtaagctcaagttactaaaggcatgcatgaatagacagagtctctctcgttcatttgcgagaaagctgcgatggcgaaagtttcttccagatgatcacttgcagctggcgtcagtggtgccattcaggctgctggacacatcgggtgctctgttttccgctcttagttgtcagtgctctgtttttcctctgttttagagttgtaactttcaaggggctggagaaacattcatttgtccactgaactggtgcaaccttattatgaagaggtctgcttcggtaca